SEQUENCE LISTING (5 sequences)

SEQ ID NO: 1 - DNA, 150 bp, Artificial Sequence
  Oligonucleotide
ggattggttggttatttgtttaaggaaggtggattaaagagagttagaatgtaggagtggtataggagtaattgaatgatgataggtatggaatagtagttgattaaaggcccaataaggtgatggaagttatgtttggtagattgatgg

SEQ ID NO: 2 - DNA, 26 bp, Artificial Sequence
  Forward Primer
ggattggttggttatttgtttaagga

SEQ ID NO: 3 - DNA, 28 bp, Artificial Sequence
  Reverse Primer
attattccactaccttcaataacaaacc

SEQ ID NO: 4 - DNA, 28 bp, Artificial Sequence
  Reverse Primer
actattccactaccttcaataacaaacc

SEQ ID NO: 5 - DNA, 28 bp, Artificial Sequence
  Reverse Primer
ataattccactaccttcaataacaaacc

(19) United States
(12) Patent Application Publication
(10) Pub. No.: US 2023/0416852 A1
(43) Pub. Date: Dec. 28, 2023

(54) METHODS FOR IDENTIFYING COMPOSITIONS FOR INHIBITING VIRAL INFECTIVITY

(71) Applicant: Meharry Medical College, Nashville, TN (US)

(72) Inventor: Bindong Liu, Brentwood, TN (US)

(73) Assignee: Meharry Medical College, Nashville, TN (US)

(21) Appl. No.: 18/024,684

(22) PCT Filed: Sep. 13, 2021
(86) PCT No.: PCT/US2021/050096

§ 371 Date: Mar. 3, 2023

Related U.S. Application Data
(60) Provisional application No. 63/077,221, filed on Sep. 11, 2020.

(51) Int. Cl. C12Q 1/70; C12Q 1/6851; G01N 33/50; C07D 501/02; A61K 31/546; A61K 31/196; A61K 31/353; A61K 31/35; A61K 31/517; A61K 31/4184; A61K 31/522; A61K 31/194; A61K 31/47

(52) U.S. Cl. CPC C12Q 1/703; C12Q 1/6851; G01N 33/502; C07D 501/02; A61K 31/546; A61K 31/196; A61K 31/47; A61K 31/35; A61K 31/517; A61K 31/4184; A61K 31/522; A61K 31/194; A61K 31/353

(57) ABSTRACT

Compositions that inhibit the HIV-1 viral infectivity factor (Vif) and methods of use thereof are provided. The disclosed compositions have inhibitory activity against Vif function and restore A3G enzymatic activity. The disclosed compositions may be used to treat and/or prevent infection and transmission of viruses (such as HIV), to inhibit the function of Vif in a cell, to inhibit viral infectivity in a cell, and to inhibit replication of a virus. Methods of identifying Vif inhibitors are also provided.

### CROSS-REFERENCE TO RELATED APPLICATION

This application cites the priority of currently pending U.S. 63/077,221 filed 11 Sep. 2020. U.S. 63/077,221 is incorporated herein by reference in its entirety.

### INCORPORATION-BY-REFERENCE OF SEQUENCE LISTING

The material in the accompanying sequence listing is hereby incorporated by reference in its entirety into this application. The accompanying file, named Sequences_218101_401018_ST25.txt, was created on and electronically submitted via EFS-Web on Sep. 13, 2021 and is 1.33 KB.

### FIELD OF THE DISCLOSURE

The present disclosure relates generally to compounds that inhibit the HIV-1 viral infectivity factor (Vif), methods of identifying Vif inhibitors, and methods of treatment and/or prevention of diseases and medical conditions, for example, human immunodeficiency virus (HIV).

### BACKGROUND

Human immunodeficiency virus, known as HIV, is a virus that damages the body's immune system, specifically the CD4 cells, often called T cells. In 2016, there were approximately 36.7 million people worldwide living with HIV/AIDS, and that number has continued to increase over time. Without an effective vaccine, treatment of infection and prevention of transmission remain the only options for patients. Although there are some 30 clinically approved drugs for the treatment of HIV infection as well as drug combinations that can effectively manage viral replication, eradication of the virus remains out of reach. A patient infected with HIV must undergo lifelong treatment and cessation of treatment almost universally results in a rebound of viremia. Moreover, the effectiveness of treatment is limited by incomplete adherence, which is often caused by drug side effects and patient complacency, and by viral resistance to therapy. According to surveys by the World Health Organization, in the past four years, 12 countries in Africa, Asia, and the Americas have surpassed acceptable levels of drug resistance against two drugs that constitute the backbone of HIV treatment: efavirenz and nevirapine (Rodriguez Mega E., Nature, July 2019). Therefore, to combat the increasing emergence of multidrug-resistant HIV-1, it is important to develop effective drugs against novel viral targets.

The HIV-1 viral infectivity factor (Vif) is an essential viral accessory protein for HIV replication (Strebel K et al. Nature 328:728-730 (1987); Fisher A G et al. Science 237:888-893 (1987)). HIV with defective Vif will result in a non-infectious virus, which makes Vif a good target for antiviral development. The primary function of Vif is to counteract APOBEC3G (Apolipoprotein B mRNA-editing enzyme-catalytic polypeptide-like 3G) (“A3G”), which is a cytidine deaminase that restricts replication of HIV (Sheehy A M et al. Nature 418:646-650 (2002)). In the absence of functional Vif, A3G is encapsulated into HIV-1 virions and causes deamination of viral cDNA cytidine to uridine. Subsequent replication of the cDNA induces lethal G to A hypermutations in the newly synthesized viral DNA, thereby rendering HIV non-infectious.

The general consensus in the HIV-A3G research field is that Vif counteracts A3G via proteasome-mediated A3G degradation, thus preventing its encapsulation into the budding virion (reviewed by Harris R S et al. Nat Rev Immunol 4:868-877 (2004)). However, studies have demonstrated that the protection provided against A3G is not absolute. Specifically, G to A hypermutations have been identified in the genomes of HIV-1 primary isolates, and the dominant mutation patterns of these hypermutations were in the GG and GA dinucleotides sequences. Since GG and GA are the hotspots of A3G mutation sites, the existence of G to A hypermutations in the genome of HIV primary isolates suggests that these mutations were induced by A3G, which in turn indicates that a certain amount of A3G molecules were present inside the virion. Therefore, in spite of the robust potential of Vif to limit A3G encapsidation, it has been discovered that budding HIV virions still contained a detectable amount of A3G, even in the presence of Vif.

Furthermore, recent studies suggest that Vif still protects HIV infectivity even after A3G encapsidation into the virion. For example, it has been demonstrated that purified wildtype virions produced in H9 cells, a CD4+ T cell line, contain A3G molecules with deaminase activity (Nowarski R et al. Nat Struct Mol Biol 15:1059-1066 (2008)). In addition, it has been shown that detectable amounts of A3G are present in wild-type HIV-1 particles produced from CD4+ T cells and peripheral blood mononuclear cells during infection (Gillick K et al. J Viral 87:1508-1517 (2013); Xu H et al. Virology 360:247-256 (2007)). Ultimately, data suggests that Vif harbors an additional mechanism to counteract A3G antiviral function even after A3G is encapsulated (Wang Y et al. Retrovirology 11, 89 (2014)).

This additional mechanism has been identified as the direct inhibition of A3G cytidine deaminase activity (“CDA”). Indeed, substantial evidence from both laboratory and clinical studies has shown that Vif further protects HIV infectivity by directly inhibiting A3G CDA in a degradation-independent manner. For example, it has been shown that A3G-induced cytidine deamination is inhibited by the expression of Vif, without the depletion of a deaminase domain, in an Escherichia coli system (Santa-Marta M et al. J Biol Chem 280:8765-8775 (2005)). In addition, it has been found that Vif inhibits A3G enzymatic activity by altering processive ssDNA scanning of the A3G (Feng Y et al. J Biol Chem doi:10.1074/jbc.M112.421875 (2013)). Furthermore, it has been reported that the A3G packaged with Vif in the virion was found to be less catalytically active than A3G encapsidated in the absence of Vif (Britan-Rosich E et al. J Mol Biol 410:1065-1076 (2011)). It has further been shown that Vif protects HIV infectivity by reducing G to A hypermutation rate induced by encapsidated A3G during HIV replication (Wang Y et at Retrovirology 11:89 (2014)). Therefore, in sum, the evidence supports the existence of an additional mechanism of Vif on counteracting A3G antiviral function by directly inhibiting A3G CDA and reducing the G to A hypermutation rate in the HIV genome.

Nonetheless, it remains unclear which Vif function is dominant in counteracting A3G antiviral function, which is crucial for antiviral drug design targeting. Previous attempts to investigate Vif function have been inadequate, as these attempts have used either Vif or A3G mutations (or deletions) and, thus, do not accurately reflect physiological conditions. In addition, the assays employed by previous studies have been insufficient for screening for a Vif inhibitor. Specifically, gel-based assays and bacterial mutation assays are not scalable, and scintillation proximity assays are not environmentally friendly. Furthermore, resonance energy transfer (FRET)-based assays cannot obtain stable and consistent results when used to measure Vif's inhibitory effect on A3G CDA.

Accordingly, there remains a need in the art for more effective screening methods to identify Vif inhibitors as well as more effective treatments for viruses, such as HIV, that inhibit the non-traditional Vif function of inhibiting A3G CDA.

### SUMMARY

The problems described above, as well as others, are addressed by the following inventions, although it is to be understood that not every embodiment of the inventions described herein will address each of the problems described above. In some embodiments, compounds that inhibit Vif function and treat and prevent infection and transmission of viruses, such as HIV, have been unexpectedly discovered. Methods for identifying compounds that can inhibit Vif function have also been unexpectedly discovered.

In a first aspect, a method for identifying a compound that inhibits Vif function is provided, comprising: providing a mixture comprising an amount of A3G, an amount of Vif, and an amount of an oligonucleotide having a CCC sequence or CC sequence; contacting the mixture with a compound to form a sample; measuring a conversion of the oligonucleotide having a CCC sequence or CC sequence in the sample to an oligonucleotide having a CCU sequence or CU sequence; and determining the compound is capable of inhibiting Vif function based on the measurement of the conversion of the oligonucleotide having a CCC sequence or CC sequence to the oligonucleotide having the CCU sequence or CU sequence.

In a second aspect, a method of making a derivative of cefixime having an inhibitory effect on HIV-1 viral infectivity factor (Vif) is provided, the method including providing cefixime; dissolving the cefixime in solvent to form a cefixime/solvent mixture; and incubating the cefixime/solvent mixture at a temperature of about 37° C. to about 110° C. for about five days to about 30 days.

In a third aspect, a derivative of cefixime is provided, the derivative obtained by the method of the second aspect.

In a fourth aspect, a pharmaceutical composition is provided including a pharmaceutically acceptable carrier and an effective amount of one or more of the following compounds: the derivative of cefixime provided in the third aspect; a compound selected from formula (I)-(IX):

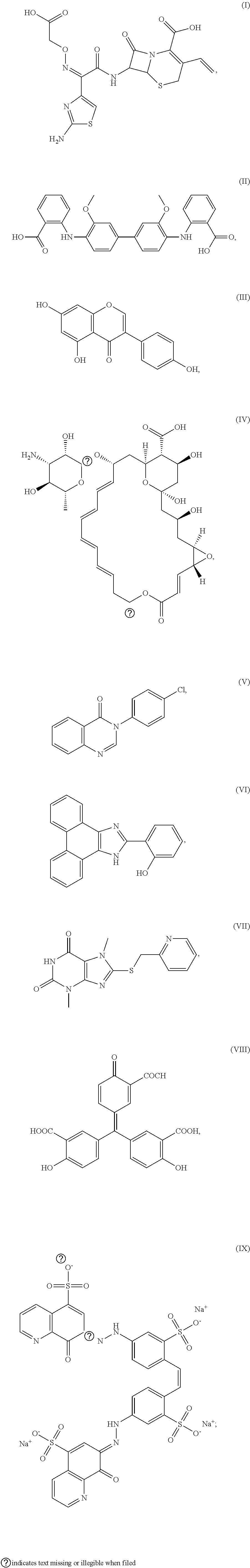

or an enantiomer, hydrate, pharmaceutically acceptable salt, stereoisomer, tautomer, or derivative thereof; or any combination thereof.

In a fifth aspect, a method for treating or preventing a virus in a subject in need thereof is provided, the method including administering to the subject an effective amount of the pharmaceutical composition provided in the fourth aspect.

In a sixth aspect, a method for treating or preventing HIV infection in a subject in need thereof is provided, the method including administering to the subject an effective amount of the pharmaceutical composition provided in the fourth aspect.

In a seventh aspect, a method for inhibiting Vif function in a cell is provided, the method including contacting the cell with an effective amount of the pharmaceutical composition provided in the fourth aspect to inhibit the function of Vif.

In an eighth aspect, a method for inhibiting viral infectivity in a cell is provided, the method including contacting the cell with an effective amount of the pharmaceutical composition provided in the fourth aspect to inhibit infectivity through restoring A3G′s capability to indue G to A hypermutation in HIV-1 viral genome.

In a ninth aspect, a method of inhibiting replication of a virus in a cell is provided, the method including contacting the cell with an effective amount of the pharmaceutical composition provided in the fourth aspect to inhibit viral replication.

### BRIEF DESCRIPTION OF THE DRAWINGS

Further features and advantages of the invention can be ascertained from the following detailed description that is provided in connection with the drawings described below:

FIG. 1 illustrates an embodiment of a qPCR-based CDA assay for measuring A3G CDA and Vif inhibitory effect on A3G CDA.

FIG. 2A is a graph showing a standard curve for measuring CCU-150 oligo that was established using a serial dilution of CCU-150 oligo as a template in the qPCR.

FIG. 2B is a graph showing mixtures of CCU-150 and CCC-150 that were subjected to the qPCR. The total amount of oligo in each reaction was: 0.1 fmol (6.7×10⁷ copies).

FIG. 2C is a graph showing that the assay activity is linearly dependent on active A3G concentration.

FIG. 2D is a graph showing different concentrations of Vif that were used to test its optimal concentration to inhibit A3G activity. The A3G activity at 150 nM Vif was set as 1 and the A3G, Vif and −150 concentrations are 200 nM, 150 nM and 0.1 fmol.

FIG. 2E is a graph showing the relative activity of the assay as DMSO was added. Different doses of DMSO were added into the assay and the relative A3G activity was measured using the assay.

FIG. 2F is a graph showing a checkboard assay of A3G versus A3G and Vif that was performed in a 96-well plate format. The checkboard assay showed a Z score of around 0.83.

FIG. 3 is a graph showing the hits from the NCI Diversity Set VI for restoring A3G CDA in the presence of Vif. Numbers I, III, V, VI, VII, and IX stand for the selected hits.

FIG. 4A shows a process of the PCR and restriction digestion-based A3G CDA assay, according to an embodiment.

FIG. 4B shows polyacrylamide gel result of the PCR and restriction digestion-based A3G CDA assay of FIG. 4A for redoxal (Compound II), cefixime (Compound VII), and C5 (cefixime derivative) and quinobene (Compound IX).

FIG. 5 is a graph showing the antiviral activity of Compound II (redoxal) and C5 in a MAGI assay.

FIG. 6A is a graph showing A3G dependent antiviral activity of quinobene and C5 in CEM cells.

FIG. 6B is a graph showing A3G independent antiviral activity of quinobene and C5 in CEM SS cells.

FIG. 6C is a graph showing the cytotoxicity of C5 and quinobene in CEM cells.

FIG. 7A is a Western blot showing that quinobene has minimal to no influence on Vif induced A3G degradation.

FIG. 7B is a Western blot showing that quinobene has minimal to no effects on A3G viral encapsidation.

FIG. 8 is a Western blot showing that redoxal (Compound II) enhances A3G expression independent of Vif, but also reduced overall Gag expression and may alter Gag processing patterns.

### DETAILED DESCRIPTION

### I. Definitions

Unless otherwise defined, all terms (including technical and scientific terms) used herein have the same meaning as commonly understood by one of ordinary skill in the art of this disclosure. It will be further understood that terms, such as those defined in commonly used dictionaries, should be interpreted as having a meaning that is consistent with their meaning in the context of the specification and should not be interpreted in an idealized or overly formal sense unless expressly so defined herein. Well-known functions or constructions may not be described in detail for brevity or clarity.

As used herein, the singular forms “a,” “an,” and “the” include plural referents unless the context clearly dictates otherwise.

Recitation of ranges of values herein are merely intended to serve as a shorthand method of referring individually to each separate value falling within the range, unless otherwise indicated herein, and each separate value is incorporated into the specification as if it were individually recited herein.

The terms “about” and “approximately” shall generally mean an acceptable degree of error or variation for the quantity measured given the nature or precision of the measurements. Typical, exemplary degrees of error or variation are within 20 percent (%), preferably within 10%, more preferably within 5% of a given value or range of values, and still more preferably within 1% of a given value or range of values. Numerical quantities given in this description are approximate unless stated otherwise, meaning that the term “about” or “approximately” can be inferred when not expressly stated.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. Terms such as “at least one of A and B” should be understood to mean “only A, only B, or both A and B.” The same construction should be applied to a longer list (e.g., “at least one of A, B, and C”). The terms “first,” “second,” and the like are used herein to describe various features or elements, but these features or elements should not be limited by these terms. These terms are only used to distinguish one feature or element from another feature or element. Thus, a first feature or element discussed below could be termed a second feature or element, and similarly, a second feature or element discussed below could be termed a first feature or element without departing from the teachings of the present disclosure.

The terms “treatment,” “treat,” and “treating” as used herein refers a course of action (such as implanting a medical device) initiated after the onset of a clinical manifestation of a disease state or condition so as to eliminate or reduce such clinical manifestation of the disease state or condition. Such treating need not be absolute to be useful. The term “in need of treatment,” as used herein, refers to a judgment made by a caregiver that a patient requires or will benefit from treatment. This judgment is made based on a variety of factors that are in the realm of a caregiver's expertise and includes the knowledge that the patient is ill, or will be ill, as the result of a condition that is treatable by a method or device of the present disclosure.

The terms “prevention,” “prevent,” “preventing,” “suppression,” “suppress,” and “suppressing,” as used herein, refer to a course of action (such as implanting a medical device) initiated prior to the onset of a clinical manifestation of a disease state or condition so as to prevent or reduce such clinical manifestation of the disease state or condition. Such preventing and suppressing need not be absolute to be useful. The term “in need of prevention” as used herein refers to a judgment made by a caregiver that a patient requires or will benefit from prevention. This judgment is made based on a variety of factors that are in the realm of a caregiver's expertise and includes the knowledge that the patient will be ill or may become ill, as the result of a condition that is preventable by a method or device of the disclosure.

In this disclosure, terms such as “administering” or “administration” include acts such as prescribing, dispensing, giving, or taking a substance such that what is prescribed, dispensed, given, or taken actually contacts the patient's body externally or internally (or both). It is specifically contemplated that instructions or a prescription by a medical professional to a subject or patient to take or otherwise self-administer a substance is an act of administration.

The terms “increase,” “enhance,” “stimulate,” and “induce” (and like terms) generally refer to the act of improving or increasing, either directly or indirectly, a function or behavior relative to the natural, expected, or average or relative to current conditions.

The terms “inhibit,” “suppress,” “decrease,” “interfere,” and/or “reduce” (and like terms) generally refer to the act of reducing, either directly or indirectly, a function, activity, or behavior relative to the natural, expected, or average or relative to current conditions. It is understood that these terms are typically in relation to some standard or expected value. In other words, they are relative, but it is not always necessary for the standard or relative value to be referenced expressly.

The term “pharmaceutically acceptable carrier” refers to one or more compatible solid or liquid fillers, diluents, or encapsulating substances that do not cause significant irritation to a human or other vertebrate animal or subject and do not abrogate the biological activity and properties of the administered compound.

The term “pharmaceutically acceptable salt” refers to salts prepared from pharmaceutically acceptable non-toxic acids or bases including inorganic acids and bases and organic acids and bases. When the compounds of the present disclosure are basic, salts may be prepared from pharmaceutically acceptable non-toxic acids including inorganic and organic acids. Suitable pharmaceutically acceptable acid addition salts for the compounds of the present invention include acetic, adipic, alginic, ascorbic, aspartic, benzenesulfonic (besylate), benzoic, boric, butyric, camphoric, camphorsulfonic, carbonic, citric, ethanedisulfonic, ethanesulfonic, ethylenediammetetraacetic, formic, fumaric, glucoheptonic, gluconic, glutamic, hydrobromic, hydrochloric, hydroiodic, hydroxynaphthoic, isethionic, lactic, lactobionic, laurylsulfonic, maleic, malic, mandelic, methanesulfonic, mucic, naphthylenesulfonic, nitric, oleic, pamoic, pantothenic, phosphoric, pivalic, polygalacturonic, salicylic, stearic, succinic, sulfuric, tannic, tartaric acid, teoclatic, p-toluenesulfonic, and the like. When the compounds contain an acidic side chain, suitable pharmaceutically acceptable base addition salts for the compounds of the present disclosure include, but are not limited to, metallic salts made from aluminum, calcium, lithium, magnesium, potassium, sodium and zinc or organic salts made from lysine, arginine, N,N′-dibenzylethylenediamine, chloroprocaine, choline, diethanolamine, ethylenediamine, meglumine (N-methylglucamine) and procaine. Further pharmaceutically acceptable salts include, when appropriate, nontoxic ammonium cations and carboxylate, sulfonate and phosphonate anions attached to alkyl having from 1 to 20 carbon atoms.

The terms “individual,” “subject,” and “patient” are used interchangeably herein, and refer to a mammal, including, but not limited to, humans, rodents, such as mice and rats, and other laboratory animals.

The terms “therapeutically effective amount” and “effective amount” refer to a dosage sufficient to treat, inhibit, prevent, reduce the severity of, or alleviate one or more symptoms of the disease being treated or to otherwise provide a desired pharmacologic and/or physiologic effect.

The term “viral infectivity” as used herein means any of the infection of a cell, the replication of a virus therein, and the production of progeny virions therefrom.

### II. Methods for Identifying Vif Inhibitors

Without being bound by any particular theory, it is believed that HIV encodes Vif to overcome A3G antiviral activity by inducing A3G degradation and inhibiting A3G cytidine deaminase activity (CDA). However, there has not been an effective method for identifying compounds that target the Vif function of inhibiting A3G CDA. Accordingly, the present disclosure provides methods of screening compounds, such as those found in combinatorial libraries, that have an undiscovered capability to inhibit Vif function and/or restore A3G enzymatic activity.

In one embodiment, a method for identifying a compound that inhibits Vif function is disclosed. The method may comprise providing a mixture of an amount of A3G and an amount of Vif for inhibiting A3G CDA. In some embodiments, the mixture includes about 50 nM to about 300 nM of A3G. In other embodiments, the mixture includes about 100 nM to about 250 nM of A3G. In still further embodiments, the mixture includes about 200 nM of A3G. In some embodiments, the mixture includes about 50 nM to 250 NM of Vif. In other embodiments, the mixture includes about 100 nM to about 200 nM of Vif. In still further embodiments, the mixture includes about 150 nM of Vif.

The mixture may further comprise an amount of an oligonucleotide having a CCC sequence. The term “oligonucleotide” refers to a short polymer composed of deoxyribonucleotides, ribonucleotides, or any combination thereof. A CCC sequence is a group of three cytosines. Therefore, the oligonucleotide having a CCC sequence comprises a group of three cytosines. In some embodiments, the oligonucleotide may have a CC sequence—a group of two cytosines. Unless specified otherwise or is clear from context, any CCC sequence may alternatively be a CC sequence.

In one embodiment, the mixture comprises at least one isolated oligonucleotide having a nucleotide sequence of SEQ ID NO: 1: GGATTGGTTGGTTATTTGTTTAAGGAAGGTGGATTAAAGAGAGTTAGAATGTAGGAGTGGTATAGG AGTAATTGAATGATGATAGGTATGGAATAGTAGTTGATTAAAGGCCCAATAAGGTGATGGAAGTTAT GTTTGGTAGATTGATGG. In some embodiments, the mixture includes about 0.01 to about 0.5 fmole of oligonucleotide having a CCC sequence. In other embodiments, the mixture includes about 0.05 to about 0.3 fmole of oligonucleotide having a CCC sequence. In still further embodiments, the mixture includes about 0.1 of oligonucleotide having a CCC sequence.

Without being bound by any particular theory, it is believed that the A3G CDA edits CCC sequences to CCU sequences. That is, A3G CDA edits a grouping of three cytosines to two cytosines and a uracil. Therefore, if the A3G of the mixture is uninhibited the mixture lacks a sufficient amount of Vif to inhibit A3G CDA), the A3G will convert the CCC sequence of the oligonucleotide to a CCU sequence. Therefore, the presence of an oligonucleotide including a CCU sequence in the mixture is indicative of A3G CDA. In contrast, the lack of presence of an oligonucleotide including a CCU sequence in the mixture is indicative of reduced, little, or no A3G CDA. As stated above, the mixture comprises Vif, which is an inhibitor of A3G CDA. As a result, the mixture should not result in substantial conversion of the CCC sequence to the CCU sequence, without a Vif-inhibiting compound.

The method may comprise contacting the mixture to a compound to form a sample. If the compound inhibits Vif function, then A3G CDA will not be inhibited. As a result, the A3G CDA will convert some or an effective amount of the CCC sequence of the oligonucleotide of the sample to a CCU sequence. On the other hand, if the compound does not inhibit Vif function, then A3G CDA will be inhibited by Vif. Therefore, without compounds that inhibit the impact of Vif on A3G CDA, some or an effective amount of the CCC sequence of the oligonucleotide of the mixture will not be converted to a CCU sequence.

The method may further comprise measuring a conversion of the CCC sequence of the oligonucleotide to a CCU sequence. This measurement may be conducted by determining a quantity of oligonucleotide including a CCU sequence present in the sample. In some embodiments, this measurement may occur via a quantitative polymerase chain reaction (qPCR), which can measure the quantity of oligonucleotide including a CCU sequence in the sample, thereby quantifying the compound's inhibitory effect of Vif. The term “qPCR,” also known as real-time PCR, means a polymerase chain reaction designed to measure the abundance of one or more specific target sequences in a sample. Primers, which are short DNA fragments containing sequences complementary to the DNA sequence to be copied are used to select and copy the target sequence. Quantitative measurements are then made of the target sequence. Techniques for quantitative PCR are well known in the art and are exemplified in the following manuscripts incorporated by reference herein: Gu Z. (2003) J. Am. Clin. Microbiol., 41: 4636-4641; Becker-Andre M.; and Hahlblock K. K. (1989) Nucleic Acids Res. 17: 9437-9446; Freeman W., et al. M. M. (1999) Biotechniques, 26: 112-122, 124-125; Lutfalla G. et al, and Uze G. (2006) Methods Enzymol. 410: 386-400; Clementi M. et al. (1993) PCR Methods Appl. 2: 191-196; Divacco S. M. (1992) Gene, 122: 313-320.

Using qPCR, the quantity of oligonucleotide including a CCU sequence present in the sample may be amplified using primers. In one embodiment, a forward primer capable of hybridizing the oligonucleotide is provided. In some embodiments, the forward primer may include the sequence: GGATTGGTTGGTTATTTGTTTAAGGA (SEQ ID NO: 2). In another embodiment, a reverse primer capable of specifically hybridizing to the CCU sequence may be provided. The reverse primer may include any one of the following sequences: 3′-ATTATTCCACTACCTTCAATAACAAACC-5′ (SEQ ID NO: 3); 3′-ACTATTCCACTACCTTCAATAACAAACC-5′ (SEQ ID NO: 4); and 3′-ATAATTCCACTACCTTCAATAACAAACC-5′ (SEQ ID NO: 5). In some embodiments, the reverse primer includes SEQ ID NO: 5. The various embodiments described herein for the forward and reverse primer ensure that the presence of an oligonucleotide including a CCU sequence may be detected and quantified in the sample. In some embodiments, the oligonucleotide may have a CU sequence—a group of a cytosine and a uracil. Unless specified otherwise or is clear from context, any CCU sequence may alternatively be a CU sequence.

SEQ ID NO: 4 may comprise a mismatched nucleotide adjacent to the adenine at the 3′-end. As used herein, “mismatched nucleotide pair” or “mismatched nucleotide pairs” or “mismatched nucleotides” refer to a pair of nucleotides contained in opposite strands of a largely complementary double strand DNA that are juxtaposed opposite to each other but comprise nucleotide pairs that are not GC or AT. Examples of mismatched nucleotide pairs are GG, CC, AA, TT, GA, GT, CA, and CT. “Mismatched nucleotide” refers to a single nucleotide that is one of the nucleotides in a mismatched nucleotide pair.

The method may further comprise determining that the compound is capable of inhibiting Vif function based on the measurement of the conversion of the CCC sequence to the CCU sequence. If the measurement indicates little or no increase in the oligonucleotide including a CCU sequence relative to a control lacking the compound (i.e., control consists of A3G, Vif, and the oligonucleotide including a CCC sequence), then it may be concluded that the compound has little to no inhibitory effect upon Vif. As the lack of CCU sequence indicates that the compound is not effective to inhibit the effect of Vif on A3G CDA. On the other hand, if the measurement indicates an increase in the oligonucleotide including a CCU sequence relative to a control lacking the compound, then it may be concluded that the compound has an inhibitory effect upon Vif. As the increase in the CCU sequence signifies that A3G CDA was present in the presence of the compound, thereby indicating that the compound inhibited Vif's negative effects on A3G CDA, and as such, has anti-HIV activity.

The method may comprise determining the effect of the compound on inhibiting Vif and/or increasing A3G CDA. The effect of the compound's degree of A3G CDA restoration and Vif inhibition. It has been determined that the specific conversion of the CCC sequence to the CCU sequence is dependent (e.g., linearly dependent) on active A3G concentration in the sample. Therefore, an increase in the amount of CCU sequence relative to a control lacking the compound correlates with A3G CDA restoration. In other words, the effect of the compound on A3G function may be quantified. The compound may increase A3G CDA function by about 5%, 10%, 15%, 20%, 25%, 35%, or greater than 30%, greater than 40%, greater than 50%, greater than 60%, greater than 65%, greater than 70%, or greater than 75% as compared to positive and negative controls.

The methods disclosed herein may be used in a high throughput format. The term “high throughput” refers to the screening of multiple separate compounds in parallel, and a large number of test compounds may be screened simultaneously or nearly simultaneously. High throughput methods may permit rapid, highly parallel biological research and drug discovery. High throughput methods are known in the art, and such methods are generally performed in multiwell plates with automated liquid handling and detection equipment. For example, using a 16, 24, 48, 96, or 384 well plate to perform an assay to test multiple samples simultaneously is considered “high throughput.” For example, the methods disclosed herein may be used in high throughput format to screen compounds to identify multiple compounds may inhibit Vif function, to identify candidates for drug design for treating or preventing HIV infection in vitro or in vivo.

### III. Kits for Identifying Vif Inhibitors

In some embodiments, a kit for identifying Vif inhibitors comprises a solution including an amount of A3G, an amount of Vif, and an amount of an oligonucleotide having a CCC sequence. The solution may be provided as a single solution or any number of solutions. In some embodiments, the solution comprises a concentration of about 50 nM to about 300 nM of A3G, about 100 nM to about 250 nM of A3G, or about 200 nM of A3G. In further embodiments, the solution comprises a concentration of about 50 nM to 250 NM of Vif, about 100 nM to about 200 nM of Vif, or about 150 nM of Vif. In some embodiments, the solution comprises a concentration of about 0.01 to about 0.5 fmole of oligonucleotide having a CCC sequence, about 0.05 to about 0.3 fmole of oligonucleotide having a CCC sequence, or about 0.1 of oligonucleotide having a CCC sequence. The kit may include additional reagents, e.g., one or more of restriction enzymes, amplification (e.g., PCR) reagents, probes and/or primers.

### IV. Compositions for Inhibiting Vif Function

The present invention provides compounds that have been identified using the methods described above. In some embodiments, the compounds have been found to be potent virus inhibitors, such as HIV inhibitors. Without being bound by any particular theory, it is believed that the disclosed compounds target and inhibit the Vif function of inhibiting A3G CDA. Advantageously, the disclosed compounds do not affect Gag expression and processing, Vif-induced A3G degradation, or A3G viral encapsidation. As such, the compounds are effective as inhibitors of Vif and to promote A3G enzymatic activity.

In one embodiment, the compounds of the present invention include cefixime. Cefixime is a semi-synthetic cephalosporin antibiotic having the following structure:

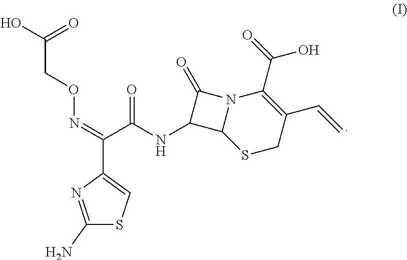 [SMILES: C=CC1=C(C(=O)O)N2C(=O)C(NC(=O)/C(=N\OCC(=O)O)c3csc(N)n3)C2SC1]

The term “cefixime” as used herein also denotes the various salt forms of cefixime including, for instance, the trihydrate salt form.

In some embodiments, the compounds of the present invention include a compound derived from cefixime. The compound derived from cefixime may be made by mixing cefixime with DMSO to form a cefixime and DMSO mixture and heating the cefixime and DMSO mixture for at least 1 hour, at least 2 hours, at least 4 hours, at least 8 hours, at least 12 hours, at least 24 hours, at least 2 days, at least 3 days, at least 4 days, at least 5 days, at least a week, or any subrange or subvalue thereof. In some embodiments, the derived compound is formed from a dry powder form of cefixime, such as by incubating and heating the dry powder form of cefixime.

In some embodiments, the derived compound may be formed by dissolving cefixime, such as the dry powder form of cefixime, in a solvent. The term “solvent” as used herein refers to a substance capable of at least partially dissolving another substance (i.e., the solute). Any suitable solvent may be used. The solvent may be a polar solvent or a non-polar solvent. The term “polar solvent” means a solvent that tends to interact with other compounds or itself through acid-base interactions, hydrogen bonding, dipole-dipole interactions, or by dipole-induced dipole interactions. Non-limiting examples of suitable polar solvents include ketones such as acetone, methyl ethyl ketone (MEK), methyl isobutyl ketone (MIBK); ethers such as tetrahydrofuran (THF), 2-methyl tetrahydrofuran, dioxane, diisopropyl ether or methyl Cert-butyl ether (MTBE); dimethylformamide (DMF); dimethylacetamide (DMA); dimethyl sulfoxide (DMSO); acetonitrile; ethyl acetate; N-methyl-2-pyrrolidone; alcohols such as methanol, ethanol, isopropanol, n-propanol, n-butanol, isobutanol, sec-butanol, or tert-butanol; water; and mixtures thereof. The term “non-polar solvent” means a solvent that is not a polar solvent. Non-polar solvents interact with other compounds or themselves predominantly through dispersion forces. Non-polar solvents interact with polar solvents mainly through dipole-induced dipole interactions or through dispersion forces. Non-limiting examples of these solvents include dichloromethane, toluene, xylene, n-heptane, octane, isooctane, cyclohexane, pentane, dioxane, and mixtures thereof.

In some embodiments, the cefixime compound may be dissolved in the solvent and incubated at a temperature of about 37° C. to about 110° C. for between about five days and about 30 days to form the cefixime powder derivative. The cefixime compound may be dissolved in the solvent and incubated at a temperature of between about 37° C. to about 90° C. for between about seven days and about 30 days to form the cefixime powder derivative. For example, the cefixime compound may be dissolved in the solvent and incubated at about 90° C. for seven days to form the cefixime powder derivative. The solvent may be DMSO, which is advantageously inexpensive and commercially available.

In still another embodiment, the compound includes redoxal (National Cancer Institute Developmental Therapeutics Program National Service Center Number (“NSC”): NSC73735), which has the following structure:

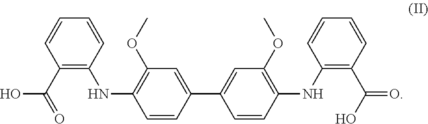 [SMILES: COc1cc(-c2ccc(Nc3ccccc3C(=O)O)c(OC)c2)ccc1Nc1ccccc1C(=O)O]

In yet another embodiment, the compound includes genistein (NSC: NSC36586), which has the following structure:

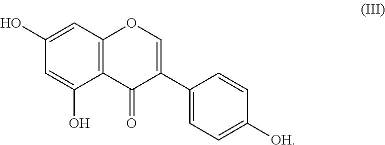 [SMILES: O=c1c(-c2ccc(O)cc2)coc2cc(O)cc(O)c12]

In other embodiments, the compound includes natamycin (NSC: SMR707), which has the following structure:

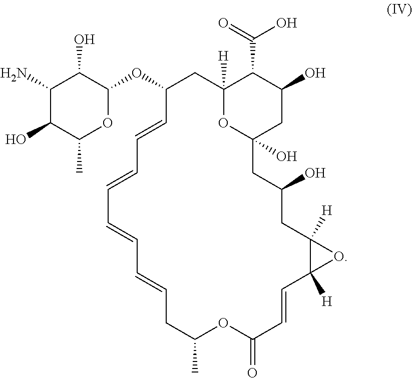 [SMILES: C[C@@H]1C/C=C/C=C/C=C/C=C/[C@H](O[C@@H]2O[C@H](C)[C@@H](O)[C@H](N)[C@@H]2O)C[C@@H]2O[C@](O)(C[C@@H](O)C[C@H]3O[C@@H]3/C=C/C(=O)O1)C[C@H](O)[C@H]2C(=O)O]

In embodiments, the compound includes a compound having one of the following structures:

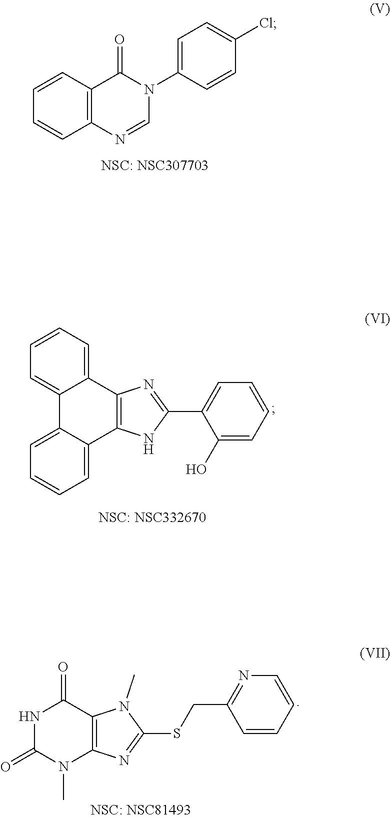 [SMILES: O=c1c2ccccc2ncn1-c1ccc(Cl)cc1 ; Oc1ccccc1-c1nc2c3ccccc3c3ccccc3c2[nH]1 ; Cn1c(SCc2ccccn2)nc2c1c(=O)[nH]c(=O)n2C]

In still embodiments, the compound includes aurintricarboxylic acid, which has the following structure:

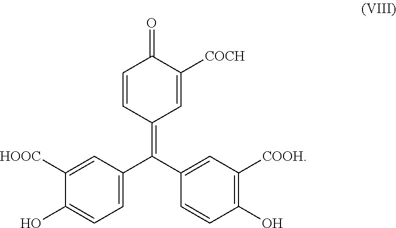

In some embodiments, the compound includes quinobene, which has the following structure:

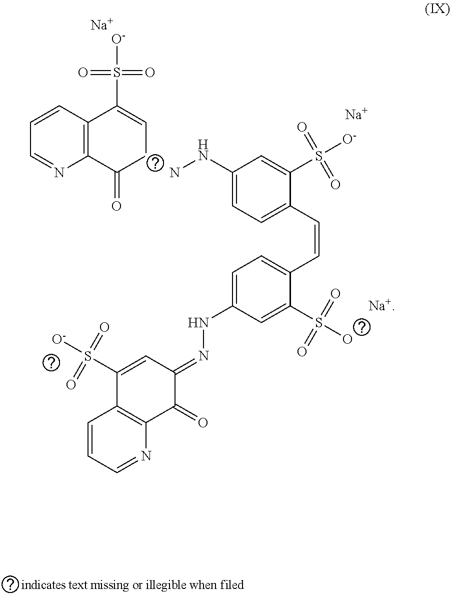

The compounds may be provided any suitable form, such as one or more of enantiomers, hydrates, polymorphs, pharmaceutically acceptable salts, esters (saturated or unsaturated), structural analogs, isomers, tautomers, and derivatives of any of the compounds disclosed above. A “derivative” may be a functional equivalent of any of the compounds, which is capable of inducing the improved pharmacological functional activity and/or behavioral response in a given subject. Exemplary chemical modifications include, but are not limited to, replacement of an alkyl group with a homolog and replacement of hydrogen by a halo group, an alkyl group, an alkoxy group, a hydroxyl group, a carboxylate, an acyl group, or an amino group.

The compounds may be racemic compounds and/or optically active isomers thereof. In this regard, some of the compounds can have asymmetric carbon atoms, and therefore, can exist either as racemic mixtures or as individual optical isomers (enantiomers) or as tautomers, for example, keto-enol and lactam-lactim tautomers. Compounds described herein that contain a chiral center include all possible stereoisomers of the compound, including compositions including the racemic mixture of the two enantiomers, as well as compositions including each enantiomer individually, substantially free of the other enantiomer.

In other embodiments, the compounds may act as a model (for example, a template) for the development of derivative compounds which are a functional equivalent of the compound and which are capable of inducing the improved pharmacological functional activity and/or behavioral response in a given subject, or in vitro.

### V. Pharmaceutical Compositions

In one embodiment, the compounds are in the form of pharmaceutical compositions. Pharmaceutical compositions are provided including one or more of the compounds, and optionally pharmaceutically acceptable diluents, preservatives, solubilizers, emulsifiers, adjuvants, excipients, and/or carriers.

To form pharmaceutically acceptable compositions suitable for administration, such compositions contain a therapeutically effective amount of one or more of the compounds. The pharmaceutical compositions are administered to a subject in an amount sufficient to deliver a therapeutically effective amount of the one or more compounds so as to be effective in the treatment and prevention methods disclosed herein. The precise dosage may vary according to a variety of factors such as, but not limited to, the subject's condition, weight, sex, and age. The selected dosage may depend upon the desired therapeutic effect, on the route of administration, and on the duration of the treatment desired. Generally, for intravenous injection or infusion, the dosage may be lower.

Alternatively, the pharmaceutical compositions may be formulated to achieve a desired concentration of the compounds at a target cell of the subject. For example, the pharmaceutical compositions may be formulated to achieve desired concentrations at one or more cells susceptible to infection including, but not limited to, dendritic cells, T cells, such as CD4+ T cells, H9 cells, CEM cells, and SupT1 cells, oral mucosa, vaginal epithelial cells, cervical epithelial cells, uterine epithelial cells, and rectal epithelial cells. In some embodiments, the composition includes an effective amount of one or more of the compounds sufficient to achieve a concentration of at least 100 nM or up to 100 μM at the one or more cells susceptible to infection. In some embodiments, the effective amount of the one or more compounds is sufficient to achieve a concentration of about 100 nM to about 50 μM at the one or more cells susceptible to infection. In another embodiment, the composition includes an effective amount of one or more of the compounds sufficient to achieve a concentration of about 50 nM to about 50 μM at the one or more cells susceptible to infection.

The pharmaceutical compositions may be formulated to be provided to the subject in any method known in the art. For instance, the pharmaceutical compositions may be formulated for administration by parenteral (for example, intramuscular, intraperitoneal, intravitreally, intravenous (IV), or subcutaneous injection), enteral, transmucosal (for example, nasal, vaginal, rectal, or sublingual), or transdermal routes of administration and can be formulated in dosage forms appropriate for each route of administration. The compositions may be formulated to be administered only once to the subject or more than once to the subject. Furthermore, when the compositions are administered to the subject more than once, a variety of regimens may be used, such as, but not limited to, once per day, once per week, once per month or once per year. The compositions may also be formulated to be administered to the subject more than one time per day. The compositions may be administered in a therapeutically effective amount to a subject. The therapeutically effective amount of the one or more compounds and appropriate dosing regimens may be identified by routine testing to obtain optimal activity, while minimizing any potential side effects. In addition, formulation for co-administration or sequential administration of other agents may be desirable.

In certain embodiments, the pharmaceutical compositions may be formulated to be administered systemically, such as by intravenous administration, or locally such as by subcutaneous injection. Typically, local administration causes an increased localized concentration of the composition which is greater than that which can be achieved by systemic administration.

The pharmaceutical compositions may further include agents which improve the solubility, half-life, absorption, etc. of the active molecule. Furthermore, the pharmaceutical compositions may further include agents that attenuate undesirable side effects and/or decrease the toxicity of the active molecule. Examples of such agents are described in a variety of texts, such as, but not limited to, Remington: The Science and Practice of Pharmacy (20th Ed., Lippincott, Williams & Wilkins, Daniel Limmer, editor).

### 1. Formulations for Parenteral Administration

The pharmaceutical compositions may be formulated for parenteral administration, for example, intramuscular, intraperitoneal, intravenous, or subcutaneous administration. In some embodiments, the compositions herein are formulated for parenteral injection, for example, in an aqueous solution. The formulation may also be in the form of a suspension or emulsion. The pharmaceutical compositions may optionally include one or more of the following for parenteral administration: diluents, sterile water, buffered saline of various buffer content (for example, Tris-HCl, acetate, phosphate), pH and ionic strength, ionic liquids, and HPβCD: and additives such as detergents and solubilizing agents (for example, TWEEN®20 (polysorbate-20) and TWEEN®80 (polysorbate-80)), anti-oxidants (for example, ascorbic acid, sodium metabisulfite), and preservatives (for example, Thimersol, benzyl alcohol) and bulking substances (for example, lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

### 2. Formulations for Enteral Administration

In some embodiments, the pharmaceutical compositions are formulated for enteral administration including oral, sublingual, and rectal delivery. In one embodiment, the compositions are administered in solid dosage form. Suitable solid dosage forms include tablets, capsules, pills, lozenges, cachets, pellets, powders, granules, or incorporation of the material into particulate preparations of polymeric compounds such as polylactic acid, polyglycolic acid, or into liposomes. In another embodiment, the compositions are administered in liquid dosage form. Examples of liquid dosage forms for enteral administration include pharmaceutically acceptable emulsions, solutions, suspensions, and syrups, which may contain other components including inert diluents; preservatives; binders; stabilizers; adjuvants such as wetting agents, emulsifying and suspending agents; and sweetening, flavoring, and perfuming agents.

Controlled release oral formulations, for example, delayed release or extended release formulations, may also be desirable. For example, the compounds may be encapsulated in a soft or hard gelatin or non-gelatin capsule or dispersed in a dispersing medium to form an oral suspension or syrup. The particles can be formed of the compound and a controlled release polymer or matrix. Alternatively, the particles can be coated with one or more controlled release coatings (for example, delayed release or extended release coatings) prior to incorporation into the finished dosage form. In still another embodiment, the compounds may be dispersed in a matrix material, which gels or emulsifies upon contact with an aqueous medium. Such matrix dispersions may be formulated as tablets or as fill materials for hard and soft capsules.

For enteral formulations, the location of release may be the stomach, the small intestine (the duodenum, the jejunem, or the ileum), or the large intestine. In some embodiments, the release will avoid the deleterious effects of the stomach environment, either by protection of the agent (or derivative) or by release of the agent (or derivative) beyond the stomach environment, such as in the intestine. To ensure full gastric resistance, a coating impermeable to at least pH 5.0 is essential. Examples of common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D™, Aquateric™, cellulose acetate phthalate (CAP), Eudragit L™, Eudragit S™, and Shellac™. These coatings may be used as mixed films.

### 3. Formulations for Topical Administration

In other embodiments, the pharmaceutical compositions are formulated for topical application. Topical dosage forms include, but are not limited to, lotions, sprays, ointments, creams, pastes, and emulsions, containing the active molecule, can be admixed with penetration enhancers and a variety of carrier materials well known in the art, such as alcohols, aloe vera gel, allantoin, glycerine, vitamin A and E oils, mineral oil, PPG2 myristyl propionate, and the like, to form alcoholic solutions, topical cleansers, cleansing creams, skin gels, skin lotions, and shampoos in cream or gel formulations. Inclusion of a skin exfoliant or dermal abrasive preparation may also be used. Such topical preparations may be applied to a patch, bandage or dressing for transdermal delivery, or may be applied to a bandage or dressing for delivery directly to the site of a wound or cutaneous injury.

### 4. Formulations for Transmucosal Administration

In some embodiments, the pharmaceutical compositions may be formulated for transmucosal administration. Transmucosal administration refers to a route of administration in which the drug is diffused through the mucous membrane. For instance, the compositions may be formulated for inhalation, nasal, oral (sublingual, buccal), vaginal, rectal, or ocular routes. Formulations for administration to the mucosa will typically be spray dried drug particles, which may be incorporated into, for example, a tablet, gel, capsule, suspension, emulsion, cream, foam, ointment, tampon, enema solution, or suppository.

### VI. Methods of Treatment and Prevention

The pharmaceutical compositions and compounds can be used, for example, to treat and/or prevent infection and transmission of viruses, such as HIV, to inhibit the function of Vif in a cell, to inhibit viral infectivity in a cell, and to inhibit replication of a virus, in vivo and in vitro.

In some embodiments, the effect of the composition on a subject is compared to a control. For example, the effect of the composition or compound on a particular symptom, pharmacologic, or physiologic indicator can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or an average determined from measuring the symptom, pharmacologic, or physiologic indicator in one or more subjects that do not have the disease or condition to be treated (for example, healthy subjects). In some embodiments, the effect of the treatment is compared to a conventional treatment that is known in the art.

In one embodiment, a method of treatment and/or prevention of infection and/or transmission of a virus in a subject in need thereof, the method including administering any of the compounds or pharmaceutical compositions to the subject in a therapeutically effective amount. Viruses that can be prevented or treated by the compositions include, but are not limited to, human immunodeficiency virus (HIV), such as human immunodeficiency virus type I (HIV-1) and human immunodeficiency virus type II (HIV-2), and other lentiviruses (e.g., simian immunodeficiency Virus (SIV), bovine immunodeficiency virus (BIV), feline immunodeficiency virus (FIV), and/or maedi-visna virus (MVV)). The method may also be useful for treatment and/or prevention of a disease and/or condition caused by the virus. For example, diseases and/or conditions that can be prevented or treated by the compositions include, but are not limited to, acquired immune deficiency syndrome (AIDS), HBV, HCV, and different forms of malignancies such as leukemia, lymphomas, myelomas, sarcomas, and tumors.

In some embodiments, the compounds or pharmaceutical compositions are used to treat or prevent infection and transmission of HIV. In this aspect, a further embodiment of the method includes co-administering an anti-HIV therapy to the subject. An anti-HIV therapy, as used herein, is any therapeutic that is useful for reducing viral load, preventing viral infection, prolonging the asymptotic phase of HIV infection, prolonging the life of a subject infected with HIV, or providing a therapeutic effect to a subject infected with HIV such as treating, inhibiting, preventing, reducing the severity of, or alleviating one or more symptoms associated with HIV. Anti-HIV therapies include, but are not limited to, nucleoside reverse transcriptase inhibitors (NRTIs) such as abacavir, emtricitabine, lamivudine, tenofovir disoproxil fumarate, and zidovudie; non-nucleoside reverse transcriptase inhibitors (NNRTIs) such as efavirenz, etravirine, nevirapine, and rilpivirine; inhibitors of HIV replication, such as protease inhibitors, e.g., atazanavir, darunavir, fosamprenavir, ritonavir, saquinavir, and tipranavir; fusion inhibitors such as enfuvirtide; CCR5 antagonists such as maraviroc; integrase inhibitors such as dolutegravir and raltegravir; post-attachment inhibitors such as ibalizumab; pharmacokinetic enhancers such as cobicistat; combination HIV medicines such as (i) abacavir and lamivudine, (ii) abacavir, dolutegravir, and lamivudine, (iii) abacavir, lamivudine, and zidovudine, (iv) atazanavir and cobicistat, (v) bictegravir, emtricitabine, and tenofovir alafenamide, (vi) darunavir and cobicistat, (vii) dolutegravir and rilpivirine, (viii) efavirenz, emtricitabine, and tenofovir disoproxil fumarate, (ix) efavirenz, lamivudine, and tenofovir disoproxil fumarate, (x) elvitegravir, cobicistat, emtricitabine, and tenofovir alafenamide fumarate, (xi) elvitegravir, cobicistat, emtricitabine, and tenofovir disoproxil fumarate, (xii) emtricitabine, rilpivirine, and tenofovir alafenamide, (xiii) emtricitabine, rilpivirine, and tenofovir disoproxil fumarate, (xiv) emtricitabine and tenofovir alafenamide, (xv) emtricitabine and tenofovir disoproxil fumarate, (xvi) lamivudine and tenofovir disoproxil fumarate, (xvii) lamivudine and zidovudine, and (xviii) lopinavir and ritonavir; cytokines; and chemokines.

The method of treatment and/or prevention includes administering to the subject any one of the compounds or pharmaceutical compositions in an amount sufficient to treat or prevent a virus, such as HIV. The method may include identifying a subject in need of such treatment or prevention. In an embodiment, the method includes delivering the compounds or pharmaceutical compositions to a site of infection in the subject. Sites of infection of a virus may include, but are not limited to, dendritic cells, T cells, such as CD4+ T cells, H9 cells, CEM cells, and SupT1 cells, oral mucosa, vaginal epithelial cells, cervical epithelial cells, uterine epithelial cells, and rectal epithelial cells. In still another embodiment, when the subject is infected or suspected to be infected with HIV, the method may include delivering the compounds or pharmaceutical compositions to an HIV competent host cell of the subject.

If, after the administration of the compounds or pharmaceutical compositions, the subject is still infected with the virus, then an optional step of the method is to continue administration of the compounds or pharmaceutical compositions.

In another embodiment, the present disclosure provides a method for inhibiting Vif function in a cell, in vitro or in vivo. As discussed above, without being bound by any particular theory, the compounds inhibit the Vif function of reducing A3G CDA. In this embodiment, the method includes contacting the cell with an effective amount of any of the compounds or pharmaceutical compositions to inhibit the function of Vif.

In still another embodiment, the present disclosure provides a method for inhibiting viral infectivity in a cell, in vitro or in vivo. The method of inhibiting viral infectivity includes contacting the cell with an effective amount of any of the compounds or pharmaceutical compositions to inhibit viral entry into the cell. For example, the compounds can act as agents for inhibiting viral entry into cells.

In yet another embodiment, the present disclosure relates to a method for inhibiting replication of any one or more of the viruses disclosed above. In this embodiment, the method includes administering any of the compounds or pharmaceutical compositions to the subject in a therapeutically effective amount to inhibit viral replication. For example, the method may include administering any of the compounds or pharmaceutical compositions to a subject infected with HIV in a therapeutically effective amount such that viral replication of the HIV is inhibited. The compounds can act as agents for inhibiting replication of the virus.

The compositions can be administered to a subject in need thereof in combination or alternation with other therapies and therapeutic agents. In some embodiments, the compositions and the additional therapeutic agent are administered separately, but simultaneously, or in alternation. The compositions and the additional therapeutic agent can also be administered as part of the same composition. In other embodiments, the compositions and the second therapeutic agent are administered separately and at different times, but as part of the same treatment regime.

The subject can be administered a first therapeutic agent 1, 2, 3, 4, 5, 6, or more hours, or 1, 2, 3, 4, 5, 6, 7, or more days before administration of a second therapeutic agent. In some embodiments, the subject can be administered one or more doses of the first agent every 1, 2, 3, 4, 5, 6 7, 14, 21, 28, 35, or 48 days prior to a first administration of second agent. The compositions can be the first or the second therapeutic agent.

The compositions and the additional therapeutic agent can be administered as part of a therapeutic regimen. For example, if a first therapeutic agent can be administered to a subject every fourth day, the second therapeutic agent can be administered on the first, second, third, or fourth day, or combinations thereof. The first therapeutic agent or second therapeutic agent may be repeatedly administered throughout the entire treatment regimen.

Exemplary molecules that may be administered with the compositions include, but are not limited to, cytokines, chemotherapeutic agents, radionuclides, other immunotherapeutics, enzymes, antimicrobials, antibiotics, antifungals, antivirals, anti-HIV therapies including, but not limited to, nucleoside reverse transcriptase inhibitors (NRTIs) such as abacavir, emtricitabine, lamivudine, tenofovir disoproxil fumarate, and zidovudie, non-nucleoside reverse transcriptase inhibitors (NNRTIs) such as efavirenz, etravirine, nevirapine, and rilpivirine, inhibitors of HIV replication, such as protease inhibitors, e.g., atazanavir, darunavir, fosamprenavir, ritonavir, saquinavir, and tipranavir, fusion inhibitors such as enfuvirtide, CCRS antagonists such as maraviroc, integrase inhibitors such as dolutegravir and raltegravir, post-attachment inhibitors such as ibalizumab, pharmacokinetic enhancers such as cobicistat, combination HIV medicines such as (i) abacavir and lamivudine, (ii) abacavir, dolutegravir, and lamivudine, (iii) abacavir, lamivudine, and zidovudine, (iv) atazanavir and cobicistat, (v) bictegravir, emtricitabine, and tenofovir alafenamide, (vi) darunavir and cobicistat, (vii) dolutegravir and rilpivirine, (viii) efavirenz, emtricitabine, and tenofovir disoproxil fumarate, (ix) efavirenz, lamivudine, and tenofovir disoproxil fumarate, (x) elvitegravir, cobicistat, emtricitabine, and tenofovir alafenamide fumarate, (xi) elvitegravir, cobicistat, emtricitabine, and tenofovir disoproxil fumarate, (xii) emtricitabine, rilpivirine, and tenofovir alafenamide, (xiii) emtricitabine, rilpivirine, and tenofovir disoproxil fumarate, (xiv) emtricitabine and tenofovir alafenamide, (xv) emtricitabine and tenofovir disoproxil fumarate, (xvi) lamivudine and tenofovir disoproxil fumarate, (xvii) lamivudine and zidovudine, and (xviii) lopinavir and ritonavir, chemokines, anti-HBV therapies including, but not limited to, entecavir, lamivudine, adefovir dipivoxil, interferon alpha-2b, pegylated interferon, telbivudine, tenofovir alafenamide, and tenofovir, anti-parasites (heiminths, protozoans), growth factors, growth inhibitors, hormones, hormone antagonists, antibodies and bioactive fragments thereof (including humanized, single chain, and chimeric antibodies), antigen and vaccine formulations (including adjuvants), peptide drugs, anti-inflammatories, ligands that bind to Toll-Like Receptors (including but not limited to CpG oligonucleotides) to activate the innate immune system, molecules that mobilize and optimize the adaptive immune system, other molecules that activate or up-regulate the action of cytotoxic T lymphocytes, natural killer cells and helper T-cells, and other molecules that deactivate or down-regulate suppressor or regulatory T-cells.

The additional therapeutic agents are selected based on the condition, disorder or disease to be treated. For example, the compositions can be co-administered with one or more additional agents that function to enhance or promote an immune response.

### VII. EXAMPLES

### Example 1

### Development of qPCR-Based CDA Assay to Quantitively Monitor A3G CDA and Vif's Inhibitory Effect on A3G CDA

### Materials and Methods

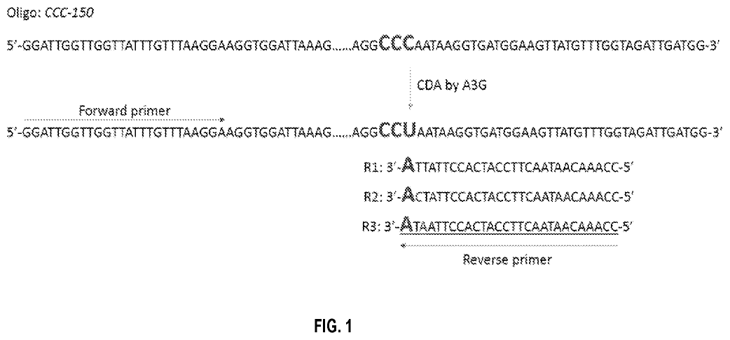

Using mismatch primer, a qPCR-based screening assay was developed to quantitively measure the inhibitory effect of Vif on A3G CDA with high sensitivity. FIG. 1 shows the schematic of the qPCR-based CDA assay for measuring Vif inhibitory effect on A3G CDA. As shown in FIG. 1, a 150 bp oligo containing a CCC sequence was used as a substrate. The A3G CDA edits the CCC sequence to predominantly to a CCU sequence. To specifically measure the C to U rate, three reverse primers R1, R2, and R3 were designed in the initial study. The reverse primers are shown in Table 1 below.

TABLE 1
Reverse Primers

| | |
|---|---|
| R1 | 3′-ATTATTCCACTACCTTCAATAACAAACC-5′ |
| | (SEQ ID NO: 3) |
| R2 | 3′-ACTATTCCACTACCTTCAATAACAAACC-5′ |
| | (SEQ ID NO: 4) |
| R3 | 3′-ATAATTCCACTACCTTCAATAACAAACC-5′ |
| | (SEQ ID NO: 5) |

It was determined that primer R3, but not primers R1 and R2, could specifically and quantitively measure the C to U conversion.

### Results

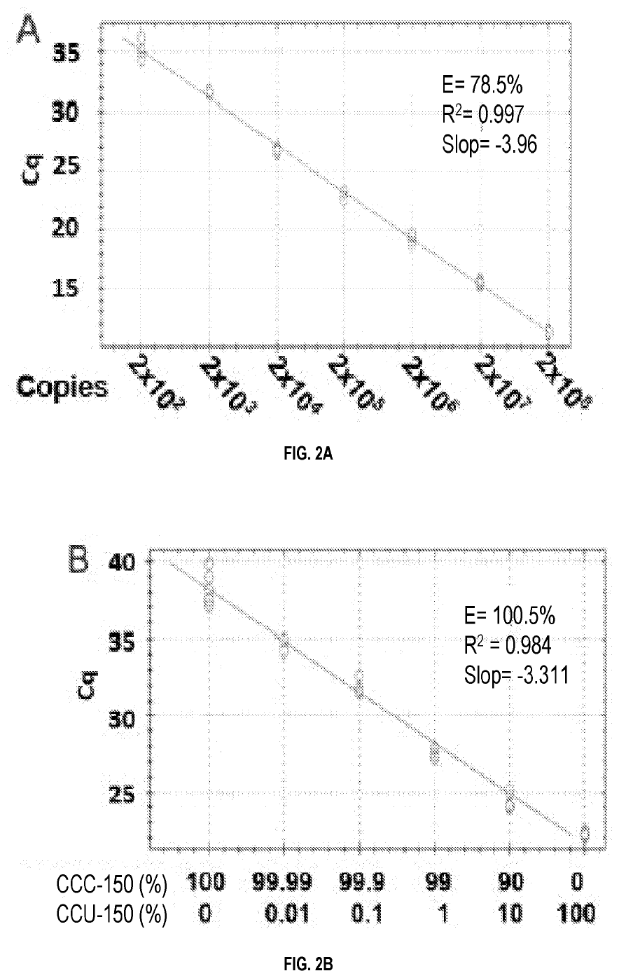

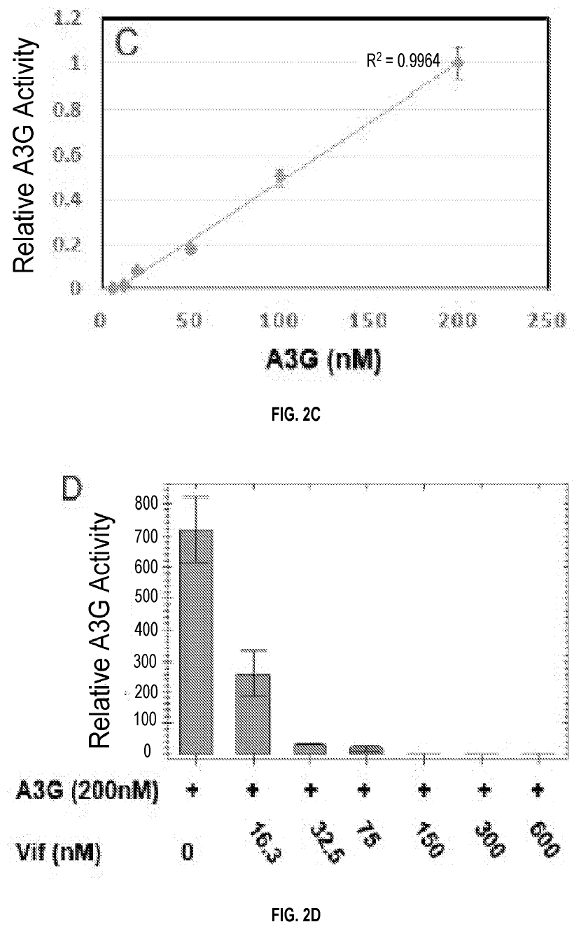

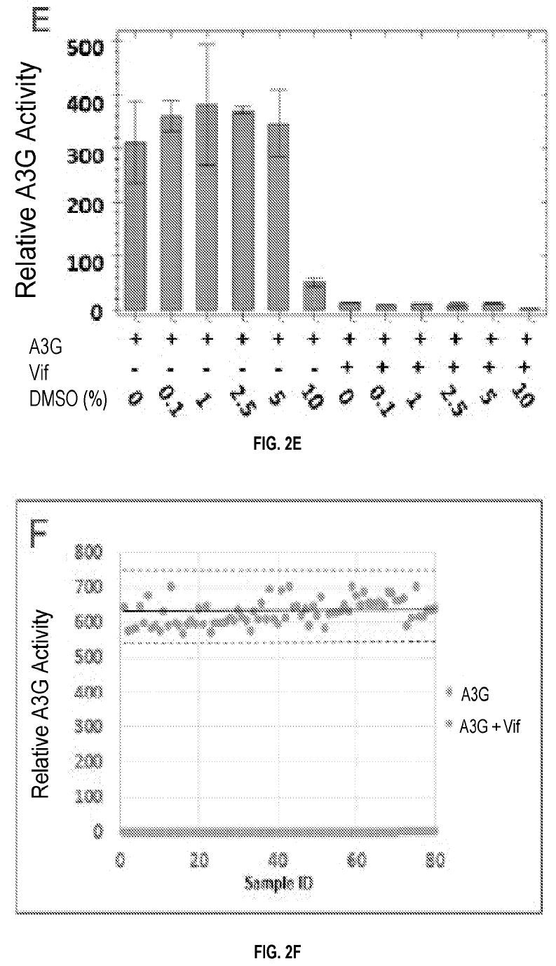

FIGS. 2A-2F show the characterization and optimization of the qPCR-based CDA assay. FIG. 2A shows a standard curve for measuring CCU-150 oligo that was established using serial dilution of CCU-150 oligo as a template in the qPCR. As shown in FIG. 2A, when oligo CCU-150 (the oligo containing a CCU sequence instead of a CCC sequence) was used to generate a standard curve, the R3 primer set was able to efficiently measure CCU-150 with a wide linear range (10⁶ range). In addition, mixing CCU-150 oligo with CCC-150 in the assay had no influence on the assay to specifically measure CCU-150, as shown in FIG. 2B. Therefore, this assay can be used to specifically measure the “CCC” to “CCU” conversion.

FIG. 2C is a graph showing the relationship between the assay activity and the active A3G concentration. Different concentrations of A3G were used in this assay to generate a standard curve. The input C00-150 was 0.1 fmol in each reaction. As shown in FIG. 20, the assay activity was linearly dependent on active A3G concentration.

FIG. 2D shows the different concentrations of Vif that were used to test its optimal concentration to inhibit A3G activity. The A3G activity at 150 nM Vif was set as 1 and the A3G, Vif and CCC-150 concentrations are 200 nM, 150 nM and 0.1 fmol. As can be seen in FIG. 2D, Vif was able to effectively inhibit A3G CDA in a dose-responsive inhibitory mode.

FIG. 2E is a graph showing the relative activity of the assay as a solvent (DMSO) was added. Different doses of the solvent were added into the assay and the relative A3G activity was measured using the assay. As shown in FIG. 2E, the assay had good tolerance up to 5% of the solvent.

FIG. 2F is a graph showing a checkboard assay of A3G versus A3G and Vif that was performed in a 96-well plate format. The checkboard assay showed a Z score around 0.83. Therefore, the qPCR-based CDA assay is robust (>0.8 Z′; >4XS/B and <10% CV).

### Example 2

### Screening of NIH Small Molecule Repository (SMR) NCI Diversity Set VI, and NCI Mech Set V With qPCR-Based CDA Assay

### Materials and Methods

Using the qPCR-based screening assay described in Example 1, the NCI Diversity Set VI, NCI Mech Set V, and NIH SMR were screened against Vif function of inhibiting A3G. The NCI Diversity Set VI was derived from almost 140,000 compounds available for distribution in the library of the NCI Developmental Therapeutics Program. The compounds of the Diversity Set VI were chosen based on structural diversity, which results in over 1,000,000 possible pharmacophores from this library. The qPCR-based CDA assay was used to screen the libraries in 96-well plates with negative controls (no A3G and Vif), positive control (no Vif), and 10 μM compounds. Hits were selected at 20% cutoff stringency (above 0.2), i.e., 20% increase in A3G CDA versus controls. Any compounds that restored A3G activity by 20% or more were selected. The positive control was set as 1.

### Results

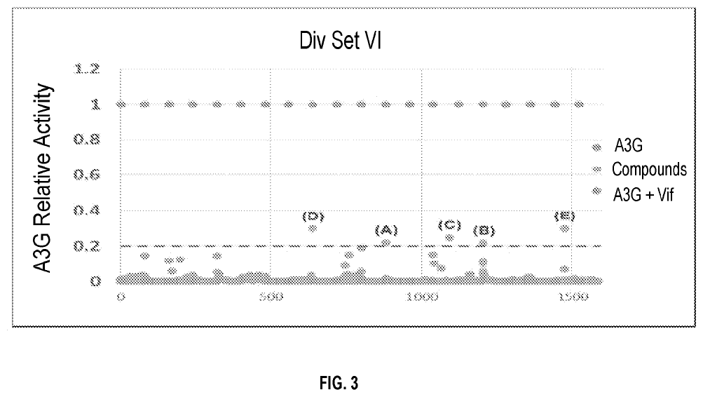

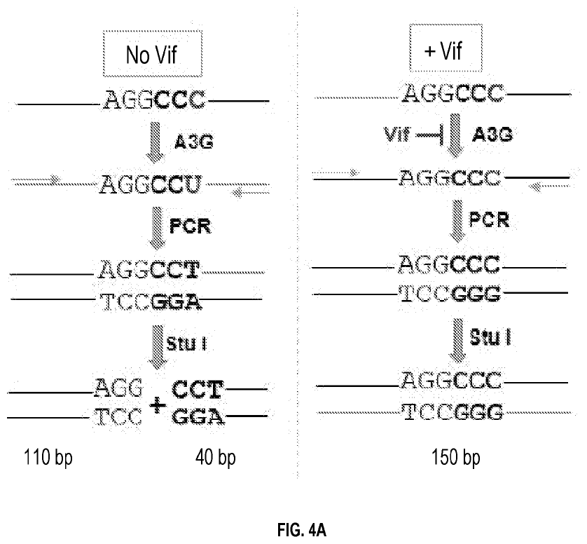

Seven hits were obtained at the cutoff stringency of 20%. Five hits were from the NCI Diversity Set VI. FIG. 3 shows the six hits from the NCI Diversity Set VI and Mech Set V. The six hits are identified as Compounds II, III, V, VI, VII and IX and have the following structures:

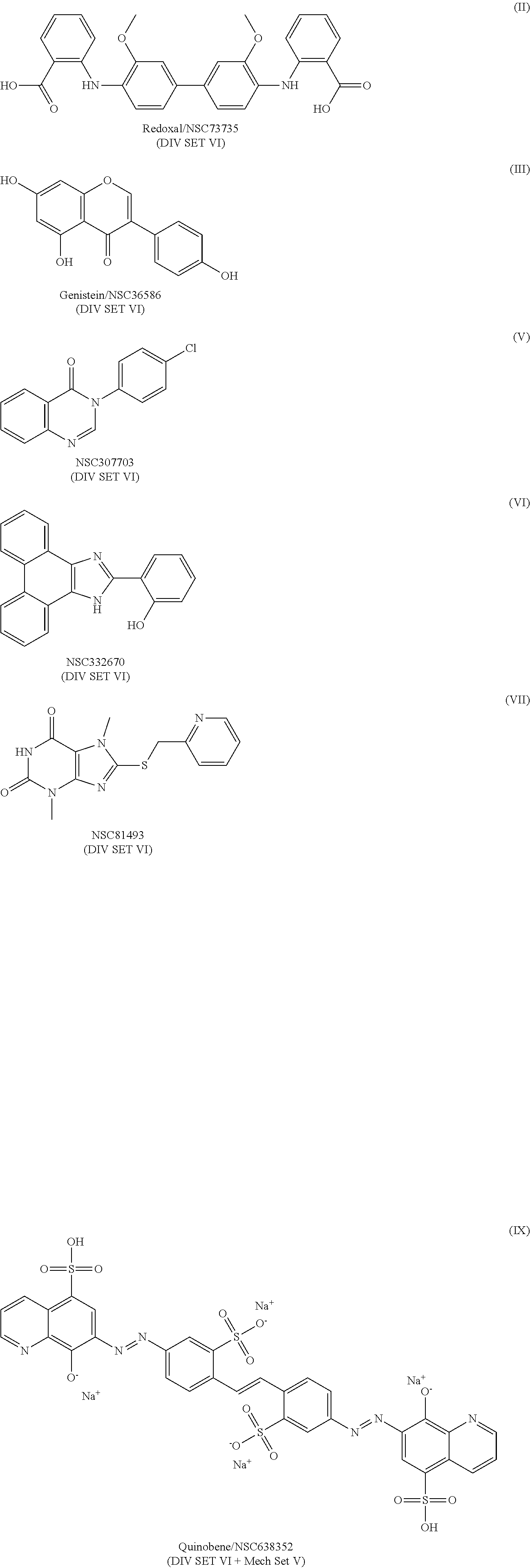

Two hits were obtained from the SMR. The two hits are identified as Compounds I and IV and have the following structures:

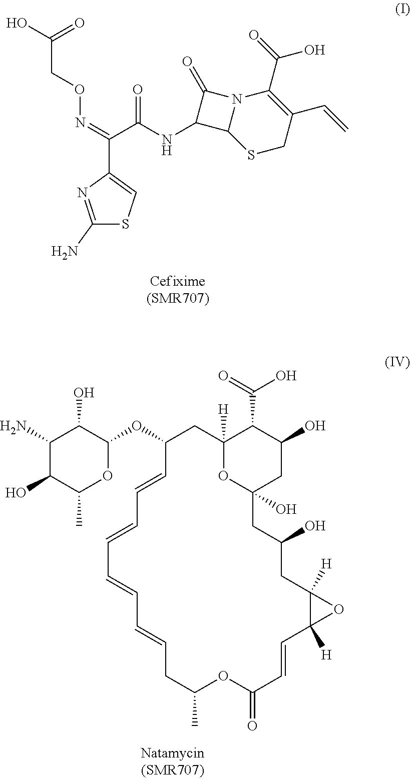 [SMILES: C=CC1=C(C(=O)O)N2C(=O)C(NC(=O)/C(=N\OCC(=O)O)c3csc(N)n3)C2SC1 ; C[C@@H]1C/C=C/C=C/C=C/C=C/[C@H](O[C@@H]2O[C@H](C)[C@@H](O)[C@H](N)[C@@H]2O)C[C@@H]2O[C@](O)(C[C@@H](O)C[C@H]3O[C@@H]3/C=C/C(=O)O1)C[C@H](O)[C@H]2C(=O)O]

A hit of Compound IX was obtained from the Mech Set V.

As shown in FIG. 3, the screening assay was highly selective against the Diversity Set VI library (hit rate about 0.3%). This demonstrates that the qPCR-based CDA assay was able to pharmacologically distinguish structurally diverse compound, in a high throughput manner.

The activity of each of Compounds III (genistein), II (redoxal), IV (natamycin) and I (cefixime) was retested in the qPCR-based CDA assay described above. In a dose-response assay, guinobene and redoxal showed high potency (EC50<6 and 12.5 μM, respectively), Genistein and natamycin did not show any activity, and Cefixime showed very low potency (EC50>500 μM) A cefixime derivative (hereinafter termed C5), which is a cefixime dry powder that was formed from incubating cefixime and DMSO at 90° C. for seven days, showed high potency (EC50<6 μM).

### Example 3

### Hit Validation for Restoring A3G CDA Using a PCR and Restriction Digestion-Based CDA Assay

### Materials and Methods

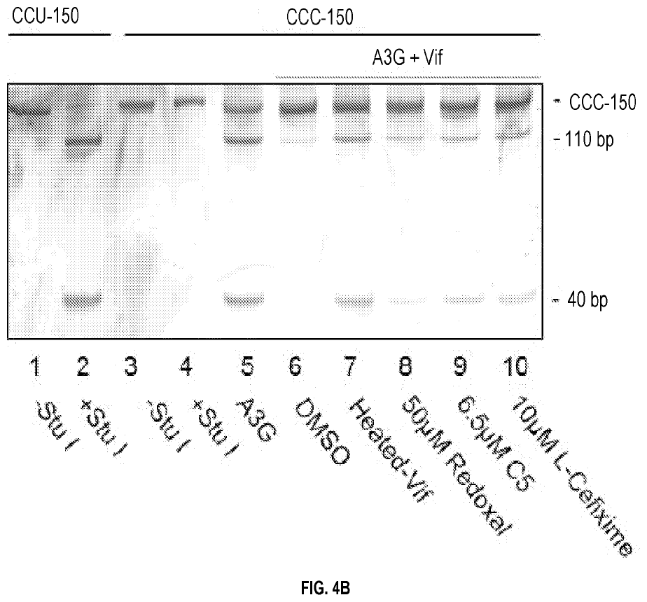

Compounds II (redoxal) and IX (quinobene) and the cefixime derivative, C5, were selected for hit validation. To eliminate the possibility of an assay-specific effect, the hits were verified by an assay using a PCR and restriction digestion-based ssDNA deaminase assay established by Nowarski et al. (Nat Struct Mol Biol 15:1059-1066 (2008)). This assay detects C to U mutation using a highly specific Stu I restriction enzyme digestion method. FIG. 4A is a schematic diagram of the PCR and restriction digestion-based CDA assay. CCC-150 is converted to CCU-150 under the enzymatic activity of A3G. As shown in FIG. 4A, the PCR reaction changes “AGGCCU” to “AGGCCT”, which is recognized by the Stu I restriction enzyme, and cuts the 150 bp oligo to two fragments, 110 bp and 40 bp. In the presence of Vif, Vif will inhibit A3G enzymatic activity, so that reduced or no CCU-150 will be produced. Therefore, less or no Stu I cutting sites will form, and no digestion will occur.

FIG. 4B shows the various treatments of the CCC-150 oligo. In the CDA reactions, the CCC-150 oligo was treated with A3G, A3G+Vif with DMSO, heat-inactivated Vif instead of active Vif (lane 7), 50 μM Compound II (redoxal), 6.5 μM C5, 10 μM Compound I (cefixime), or 5 μM compound IX (quinobene). The CDA products were subjected to PCR followed by Stu I digestion. The digestion products were applied to a 10% polyacrylamide gel for separation. SYBR Gold was used to visualize the bands. L-Cefixime refers to Compound I identified above. As a control, the CCU-150 oligo was efficiently cut into 110 bp and 40 bp by Stu I (lane 2) and the CCC-150 oligo was cut without A3G treatment (lane 4).

### Results

FIG. 4B also shows the results of the PCR and restriction digestion-based A3G CDA assay. As shown in FIG. 4B, some oligo CCC-150 with A3G was cut into 110 bp and 40 bp fragment (lanes 5 & 13). While adding Vif protein into the system dramatically reduced the digestion products (lanes 6 & 14), heat-inactivated Vif did not inhibit A3G enzymatic activity. 50 μM Compound II (redoxal), 6.5 μM C5, 10 μM Compound I (L-cefixime), and 5 uM Compound IX (quinobene) dramatically restored the digestion product. Therefore, the PCR and restriction digestion-based CDA assay further confirmed that Compound II (redoxal), C5, Compound I (L-cefixime), and Compound IX (quinobene) have the inhibitory effect against the Vif function of inhibiting A3G CDA and further demonstrated the specificity of the qPCR-based CDA assay described in Example 1.

### Example 4

### Hit Validation Using MAGI Assay

### Materials and Methods

MAGI assay is a classical method for measuring HIV infectivity (Platt E J et at J Virol 72:2855-2864 (1998)). The assay involves HIV infecting a reporter cell line, the TZM-bl cell line (Platt E J et at J Virol 72:2855-2864 (1998); Derdeyn C A et al. J Virol 74:8358-8367 (2000); Wei X et al. Antimicrob Agents Chemother 46:1896-1905 (2002)). The TZM-bl cell line is a Hela cell derivative that expresses CD4, rendering the cells permissive to HIV-1 infection. These cells also contain an integrated LTR-luciferase and β-galactosidase reporter genes, resulting in expression of firefly luciferase and β-galactosidase protein following HIV-1 integration and subsequent expression of the viral transactivator, Tat. A MAGI assay was used to test the potency of Compound II (redoxal) and C5 against HIV infectivity.

300 ng of p24 content of HIV IIIB virus was used to infect TZM-bl cells. Different doses of Compound II (redoxal) and C5 were used to treat the infected cells. DMSO treatment was used as a control. Two days post-infection, cells were fixed and β-galactosidase was stained to visualize the infected cells.

### Results

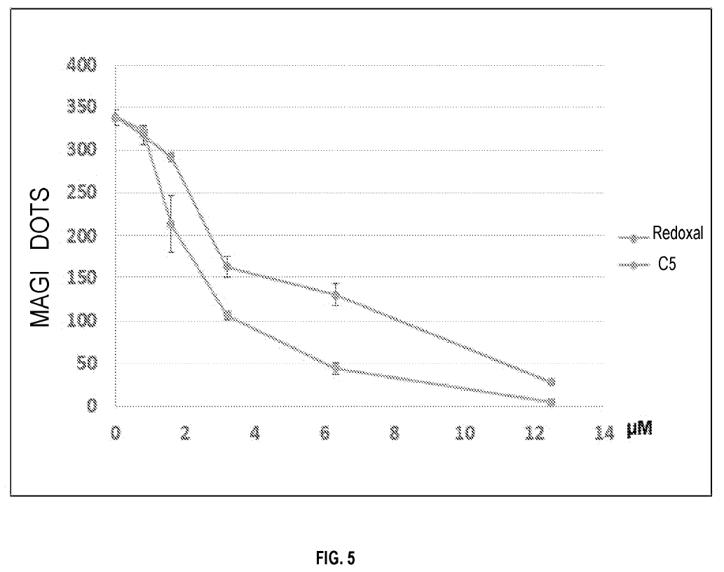

FIG. 5 shows the antiviral activity of Compound II (redoxal) and C5 in the MAGI assay. As shown in FIG. 5, both C5 and Compound II (redoxal) showed potent antiviral activity. The IC50 of Compound II (redoxal) and C5 are <4 μM and <2 μM respectively, which is consistent with the previous report that the IC50 of redoxal in PBMC was around 0.6-3 μM (Pery E et al. Virology 484:276-287 (2015)).

### Example 5

### Hit Validation of A3G-Dependent Antiviral Effects Using CD4+T, CEM-GFP, and CEM SS Cell Lines

### Materials and Methods

As discussed above, Vif functions as an inhibitor to A3G antiviral function. Therefore, a Vif specific inhibitor should only inhibit HIV replication in the presence of A3G. The A3G-dependent antiviral effects of the hits were tested using CD4+T, CEM-GFP, and CEM SS cell lines. CEM-GFP cells contain A3G protein expression, while CEM SS cells do not. The Vif specific inhibitor should show high anti-HIV potency in CEM-GFP cells (and not in CEM SS cells). The CEM-GFP cell line expresses GFP upon HIV infection driven by HIV NL4-3 LTR. It can be used to measure HIV replication (Gervaix A, et at Proc Natl Acad Sci USA. 1997; 94 (9):4653-8.)

Different doses of C5 and Quinobene were used to treat H9 and SupT1 cells. Two hours post-treatment, HIV-IIIB virus, was used to infect the H9 and SupT1 cells. The viral culture supernatant was sampled every other day and subject to MAGI to measure infectivity. Presto Blue Cytotoxicity Assay was used to measure the cytotoxicity of C5 in H9 and SupT1 cells. In this assay, live cells show 586 nm fluoresce red. The higher the fluorescence, the more viable the cells are. Therefore, 100% cell death should result in no fluorescence.

### Results

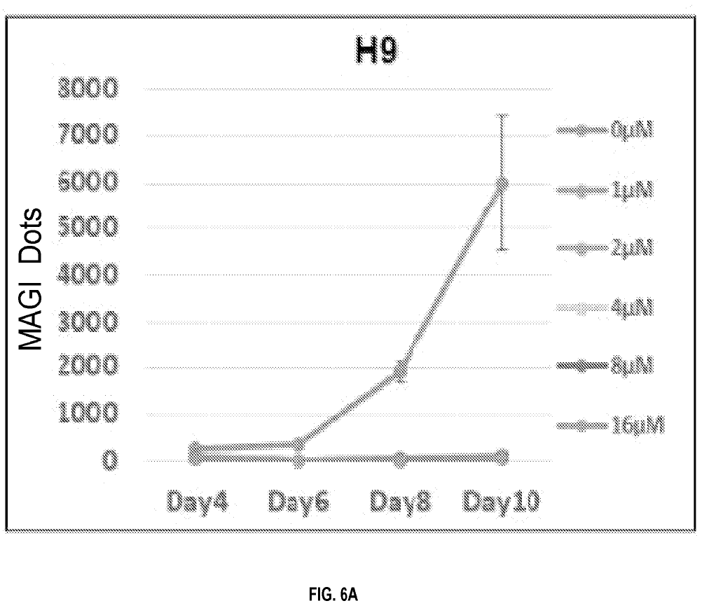

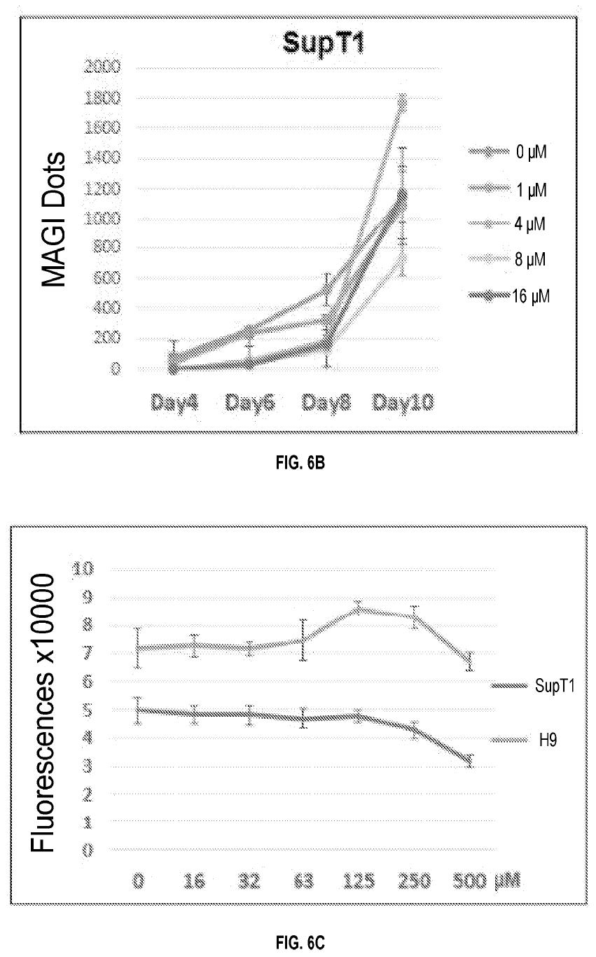

FIGS. 6A-6C show the A3G dependent antiviral activity and toxicity of C5 and quinobene. As shown in FIG. 6A, quinobene and C5 showed high potency on inhibiting HIV IIIB replication (quinobene IC50: <750 nM; C5: IC50<6 μM). In FIG. 6B, HIV-1 NL4-3-GFP was used to infect CEM SS cell line. As can be seen in FIG. 6B, in CEM SS cells, C5 showed similar moderate antiviral activity (IC50 about 5 μM) as in the CEM-GFP cells. However, quinobene lost its antiviral function in CEM SS cells comparing to that in CEM-GFP cells. This data suggests that the antiviral function of Quinobene is A3G dependent. FIG. 6C shows the cytotoxicity of C5 and quinobene in CEM cells. As shown in FIG. 6C, C5 showed almost no toxicity at up to 400 μM. The IC50 of quinobene is about 50 uM.

### Example 6

### Role of Quinobene on A3G Degradation and Viral Encapsidation

### Materials and Methods

In this example, it was determined whether C5 influences Vif induced A3G degradation and viral encapsidation. An A3G expression vector was co-transfected with Vif, or with the empty vector as control. The A3G expression vector was also co-transfected with either a wild-type viral construct HXB2, Vif deficient viral construct HXB2ΔVif, or pcDNA3.1 (as a control) into 293T cells. Immediately following transfection, 5 μM quinobene was used to treat the 293T cells. After 48 hours post-transfection, culture supernatants were subjected to ultracentrifuge to pellet down the virus. Viral lysates were analyzed by Western blot.

### Results

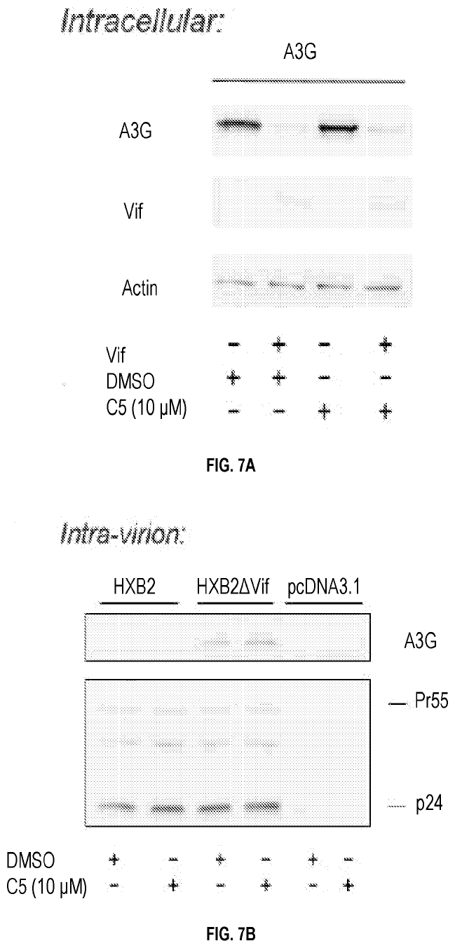

FIGS. 7A and 7B are Western blots showing the role of quinobene on A3G degradation and viral encapsidation. As can be seen in FIG. 7A, 5 μM quinobene had no influence on Vif induced A3G degradation. As shown in FIG. 7B, quinobene showed no effects on A3G viral encapsidation. Thus, it is believed that quinobene inhibits the effect of Vif on A3G CDA rather than A3G degradation and viral encapsidation.

### Example 7

### Role of Compound II (Redoxal) on A3G Stability and Viral Encapsidation

### Materials and Methods

An A3G expression vector was co-transfected with Vif, or with the empty vector as control. The A3G expression vector was also co-transfected with either a wild-type viral construct HXB2, Vif deficient viral construct HXB2ΔVif, or pcDNA3.1 (as a control) into 293T cells. Immediately following transfection, 5 μM Compound II (redoxal) was used to treat the 293T cells. After 48 hours post-transfection, culture supernatants were subjected to ultracentrifuge to pellet down the virus. Viral lysates were analyzed by Western blot.

### Results

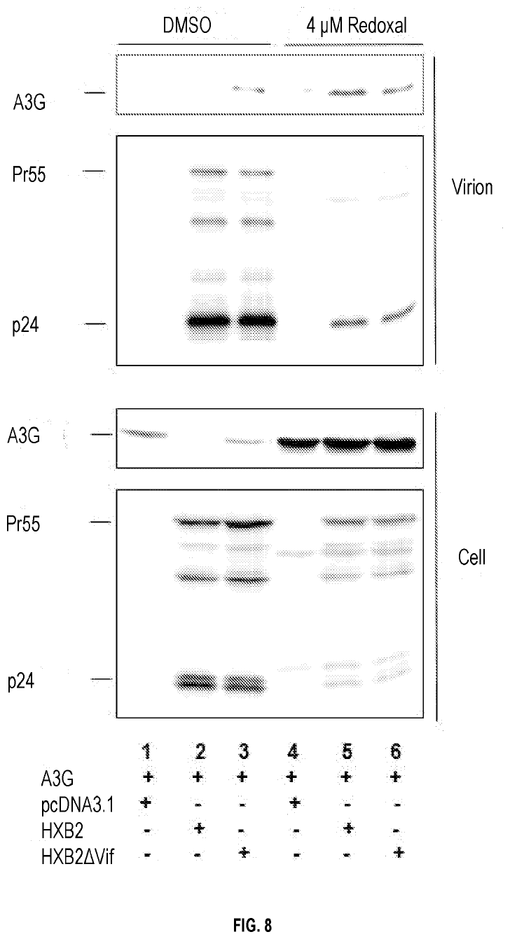

FIG. 8 is a Western blot showing the role of Compound II (redoxal) on A3G stability and viral encapsidation. As shown in FIG. 8, Compound II (redoxal) not only enhances A3G expression independent of Vif (Cell: lane 1 vs lane 4), but also reduces overall Gag expression (Cell: lane 2 vs lane 5; lane 3 vs lane 6), and may alter Gag processing patterns (Cell: lane 2 vs lane 5, lane 3 vs lane 6).

### Example 8

### Quinobene Enhances G to A Hypermutation Rate in HIV-1 Viral cDNA

### Materials and Methods

The hallmark of A3G antiviral function is to induce G to A hypermutation in HIV cDNA. Therefore, it is very important to evaluate whether quinobene restores A3G's capability to induce G to A hypermutation rates in HIV cDNA. To do this, SupT1 cells were infected with HIV IIIB (350 ng p24)+DMSO or HVI IIIB (350 ng p24)+1 μM quinobene for 6 hours. After infection, DNA was isolated using a DNeasy Blood and Tissue DNA isolation kit (QIAGEN). A 199-bp DNA fragment covering a portion of LTR of HIV-1 was amplified with Taq DNA polymerase using the primers IIIB-F (5′-CTGATATCGAGCTTGCTACAA) and HIV-1-R (5′-TGAGGCTTAAGCAGTGGGTT). The PCR products were purified by QIAquick PCR Purification Kit (Qiagen) and sent to GENEWIZ, Inc (South Plainfield, NJ) for G to A hypermutation analysis using their Amplicon-EZ service, a next-generation sequencing-based sequencing service, allowed detection of low-frequency variants quantitatively. The G to A hypermutation rate was calculated using CLC Genomic Workbench.

### Results

In Table 2, below, the left column shows the nucleotide position with potential for A3G related G to A hypermutation change; the middle and left one shows the percentage of G to A hypermutation changes.

TABLE 2
The A3G related G to A hypermutation rate in viral cDNA.

| | Untreated | Quino treated |
|---|---|---|
| Position | % | % |
| 22 | 3.79 | 5.51 |
| 23 | 0.32 | 0.27 |
| 35 | 1.57 | 1.92 |
| 36 | 2.58 | 3.87 |
| 37 | 0.37 | 0.43 |
| 47 | 1.21 | 1.84 |
| 48 | 0.28 | 0.38 |
| 51 | 0.13 | 0 |
| 56 | 0.35 | 0.53 |
| 61 | 4.5 | 6.47 |
| 62 | 0.15 | 0 |
| 65 | 3.88 | 5.65 |
| 68 | 0.33 | 0.38 |
| 71 | 1.73 | 2.13 |
| 72 | 1.73 | 2.1 |
| 73 | 0.24 | 0.28 |
| 78 | 0.22 | 0.28 |
| 126 | 5.65 | 8.57 |
| 127 | 0.33 | 0.33 |
| 136 | 1.11 | 1.39 |
| 157 | 5.76 | 8.43 |
| 158 | 0.16 | 0.19 |
| 168 | 0.4 | 0.6 |
| 177 | 2.87 | 4.16 |
| 178 | 2.36 | 2.37 |
| Total: | 42.02% | 58.08% |
| Average | 1.68% | 2.32% |

As shown in Table 2, after quinobene treatment, 22 out of 26 (84.6%) positions showed G to A hypermutation rate increases; the total G to A hypermutation rate in the 26 positions increased from 42.2% to 58%. The average for each position increased from 1.68% to 2.31%. The Paired t-test was used to calculate the p=0.0016. Similar results were obtained from three independent experiments. Therefore, the quinobene treatment significantly enhanced G to A hypermutation rate in HIV cDNA.

It is to be understood that any given element of the disclosed embodiments of the invention may be embodied in a single structure, a single step, a single substance, or the like. Similarly, a given element of the disclosed embodiment may be embodied in multiple structures, steps, substances, or the like.

The foregoing description illustrates and describes the processes, machines, manufactures, compositions of matter, and other teachings of the present disclosure. Additionally, the disclosure shows and describes only certain embodiments of the processes, machines, manufactures, compositions of matter, and other teachings disclosed, but, as mentioned above, it is to be understood that the teachings of the present disclosure are capable of use in various other combinations, modifications, and environments and is capable of changes or modifications within the scope of the teachings as expressed herein, commensurate with the skill and/or knowledge of a person having ordinary skill in the relevant art. The embodiments described hereinabove are further intended to explain certain best modes known of practicing the processes, machines, manufactures, compositions of matter, and other teachings of the present disclosure and to enable others skilled in the art to utilize the teachings of the present disclosure in such, or other, embodiments and with the various modifications required by the particular applications or uses. Accordingly, the processes, machines, manufactures, compositions of matter, and other teachings of the present disclosure are not intended to limit the exact embodiments and examples disclosed herein. Any section headings herein are provided only for consistency with the suggestions of 37 C.F.R. § 1.77 or otherwise to provide organizational queues. These headings shall not limit or characterize the invention(s) set forth herein.

What is claimed is:

1. A method of making a derivative of cefixime having an inhibitory effect on HIV-1 viral infectivity factor (Vif), comprising:
providing cefixime;
dissolving the cefixime in a solvent to form a cefixime/solvent mixture; and
incubating the cefixime/solvent mixture at a temperature of about 37° C. to about 110° C. for about five days to about 30 days to form the derivative of cefixime having an inhibitory effect on Vif.

2. The method of claim 1, wherein the cefixime/solvent mixture is incubated at a temperature of about 90° C. for about seven days.

3. The method of any one of claims 1 and 2, wherein the solvent a polar solvent.

4. The method of any one of claims 1-3, wherein the solvent is dimethyl sulfoxide (DMSO).

5. A derivative of cefixime obtained by the method of any one of claims 1-4.

6. A pharmaceutical composition, comprising:
a therapeutically effective amount of one or more of the following compounds:
the derivative of cefixime according to claim 5;
a compound selected from formula (I)-(IX): 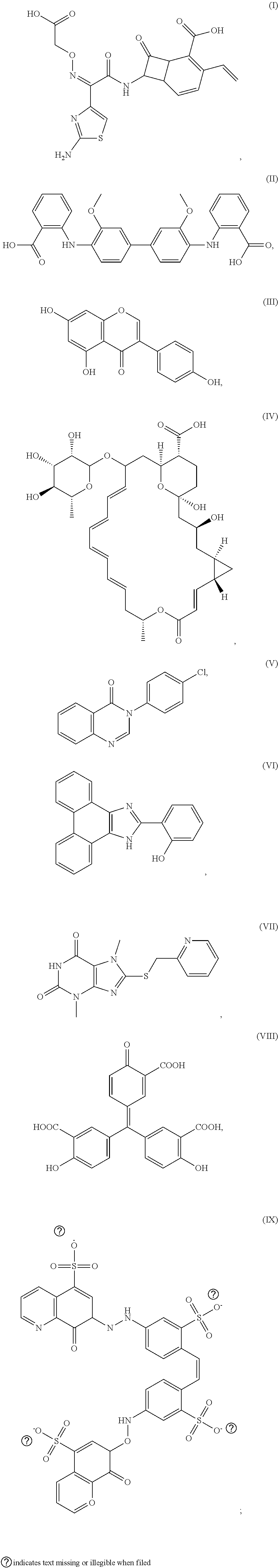
an enantiomer, hydrate, pharmaceutically acceptable salt, stereoisomer, tautomer, or derivative thereof; or
any combination thereof.

7. The pharmaceutical composition of claim 6, wherein the composition is formulated for parenteral administration.

8. The pharmaceutical composition of claim 7, wherein the parenteral administration comprises intramuscular, intraperitoneal, intravenous, or subcutaneous administration.

9. The pharmaceutical composition of claim 6, wherein the composition is formulated for trans mucosal administration.

10. The pharmaceutical composition of claim 6, wherein the composition is formulated for topical delivery.

11. The pharmaceutical composition of claim 6, wherein the composition is formulated in a dosage form selected from the group consisting of: tablet, capsule, injectable, transdermal, sublingual, cream, gel, foam, ointment, tampon, enema solution, dentifrice, gum, film, spray, lozenge, paste, gel, mouthwash, powder, tooth soap, suppository, and a combination thereof.

12. The pharmaceutical composition of any one of claims 6-11, wherein the therapeutically effective amount is sufficient to achieve a concentration of about 1 μM to about 100 μM of the one or more compounds at a target cell.

13. The pharmaceutical composition of any one of claims 6-12, wherein the therapeutically effective amount is sufficient to achieve a concentration of about 5 μM to about 50 μM of the one or more compounds at the target cell.

14. The pharmaceutical composition of any one of claims 12-13, wherein the target cell is selected from the group consisting of: dendritic cells, CD4+ T cells, CEM cells, H9 cells, SupT1 cells, oral mucosa, vaginal epithelial cells, cervical epithelial cells, uterine epithelial cells, rectal epithelial cells, and a combination thereof.

15. The pharmaceutical composition of any one of claims 6-14, wherein the therapeutically effective amount is sufficient to inhibit Vif function in the target cell.

16. The pharmaceutical composition of any one of claims 6-15, wherein the therapeutically effective amount is sufficient to reduce entry of human immunodeficiency virus (HIV) into the target cell.

17. The pharmaceutical composition of any one of claims 6-16, wherein the effective amount is sufficient to reduce HIV replication at the target cell.

18. The pharmaceutical composition of any one of claims 6-17, wherein the pharmaceutical composition is non-cytotoxic.

19. The pharmaceutical composition of any one of claims 6-18, further comprising an anti-HIV agent selected from the group consisting of: a nucleoside reverse transcriptase inhibitor (NRTIs), abacavir, emtricitabine, lamivudine, tenofovir disoproxil fumarate, zidovudine, a non-nucleoside reverse transcriptase inhibitor (NNRTIs), efavirenz, etravirine, nevirapine, rilpivirine, an inhibitor of HIV replication, a protease inhibitor, atazanavir, darunavir, fosamprenavir, ritonavir, saquinavir, tipranavir, a fusion inhibitor, enfuvirtide, a CCR5 antagonist, maraviroc, an integrase inhibitor, dolutegravir, raltegravir, a post-attachment inhibitor, ibalizumab, a pharmacokinetic enhancer, cobicistat, abacavir, lamivudine, dolutegravir, zidovudine, bictegravir, emtricitabine, tenofovir alafenamide, elvitegravir, lopinavir, a cytokine, a chemokine, and a combination thereof.

20. A method for treating or preventing a virus in a subject in need thereof, comprising:
administering to the subject an effective amount of the pharmaceutical composition of any one of claims 6-19.

21. The method of claim 20, wherein the virus is HIV.

22. The method of any one of claims 20-21, wherein the administering step comprises delivering the pharmaceutical composition to a site of infection in the subject.

23. The method of any one of claims 20-22, wherein the administering step comprises delivering the pharmaceutical composition to an HIV competent host cell of the subject.

24. A method for treating or preventing HIV infection in a subject in need thereof, comprising:
administering to the subject an effective amount of the pharmaceutical composition of any one of claims 6-19.

25. A method for inhibiting Vif function in a cell, comprising:
contacting the cell with an effective amount of the pharmaceutical composition of any one of claims 6-19 to inhibit the function of Vif.

26. A method for inhibiting viral infectivity in a cell, comprising:
contacting the cell with an effective amount of the pharmaceutical composition of any one of claims 6-19 to inhibit viral entry into the cell.

27. The method of claim 26, wherein the viral infectivity results from HIV.

28. A method of inhibiting replication of a virus in a cell, comprising:
contacting the cell with an effective amount of the pharmaceutical composition of any one of claims 6-19 to inhibit viral replication.

29. The method of claim 28, wherein the virus is HIV.

30. The method of any one of claims 25-29, wherein the cell is selected from the group consisting of: dendritic cells, CD4+ T cells, H9 cells, CEM cells, SupT1 cells, oral mucosa, vaginal epithelial cells, cervical epithelial cells, uterine epithelial cells, rectal epithelial cells, and combinations thereof.

31. The method of any one of claims 25-30, wherein the cell is selected from the group consisting of: CD4+ T cells, CEM cells, H9 cells, SupT1 cells, and combinations thereof.

32. A use of the pharmaceutical composition of any one of claims 6-19 in the manufacture of a medicament for the treatment or prevention of HIV.

33. A use of the pharmaceutical composition of any one of claims 6-19 in the treatment or prevention of HIV.

34. A method for identifying a compound that inhibits Vif function, comprising:
providing a mixture comprising
an amount of A3G,
an amount of Vif, and
an amount of an oligonucleotide having a CCC sequence or a CC sequence;
contacting the mixture with a compound to form a sample;
measuring a conversion of the oligonucleotide having a CCC sequence or a CC sequence in the sample to an oligonucleotide having a CCU sequence or a CU sequence; and
determining the compound is capable of inhibiting Vif function based on the measurement of the conversion of the oligonucleotide having a CCC sequence or a CC sequence to the oligonucleotide having the CCU sequence or the CU sequence.

35. The method of claim 34, wherein the measuring the conversion of the oligonucleotide having a CCC sequence or a CC sequence to the oligonucleotide having the CCU sequence or the CU sequence comprises measuring an amount of the oligonucleotide having the CCU sequence or CU sequence present in the sample.

36. The method of claim 34 or 35, wherein measuring the conversion of the oligonucleotide having a CCC sequence or CC sequence to the oligonucleotide having the CCU sequence or the CU sequence comprises conducting a quantitative polymerase chain reaction (qPCR) on the sample, and wherein the qPCR comprises at least one reverse primer capable of specifically hybridizing to a CCU sequence or CU sequence.

37. The method of claim 36, wherein the qPCR indicates an amount of the oligonucleotide having the CCU sequence or CU sequence that is present in the sample.

38. The method of claim 36 or 37, wherein the reverse primer comprises SEQ ID NO: 4 or SEQ ID NO: 5, wherein the SEQ ID NO:4 or the SEQ ID NO: 5 comprise an adenine at the 3′-end.

39. The method of any one of claims 36-38, wherein the forward primer comprises SEQ ID NO: 2.

40. The method of any one of claims 34-39, wherein an increase in an amount of the oligonucleotide having the CCU sequence or CU sequence relative to a control sample lacking the compound indicates the compound can inhibit Vif function.

41. The method of any one of claims 34-40, wherein an increase in an amount of the oligonucleotide having the CCU sequence or CU sequence relative to a control sample lacking the compound indicates the compound can restore A3G cytidine deaminase activity (CDA).

42. The method of claim 41, wherein the compound increases A3G CDA function by about 5% to about 10% compared to the control sample lacking the compound.

43. The method of claim 41, wherein the compound increases A3G CDA function by from about 5% to about 30% compared to the control sample lacking the compound.

44. The method of claim 41, wherein the compound increases A3G CDA function by about 10% compared to the control sample lacking the compound.

45. The method of claim 41, wherein the compound increases A3G CDA function by about 25% compared to the control sample lacking the compound.

46. The method of claim 41, wherein the compound increases A3G CDA function by about 30% compared to the control sample lacking the compound.

47. The method of claim 41, wherein the compound increases A3G CDA function by greater than 30% compared to the control sample lacking the compound.

48. The method of any one of claims 34-47, wherein an increase in an amount of the oligonucleotide having the CCU sequence or CU sequence relative to a control sample lacking the compound indicates the compound has anti-HIV activity.

49. The method of any one of claims 34-48, wherein an increase in an amount of the oligonucleotide having the CCU sequence or CU sequence relative to a control sample lacking the compound correlates with inhibition of Vif function.

50. The method of any one of claims 34-40, wherein an increase in an amount of the oligonucleotide having the CCU sequence or CU sequence relative to a control sample lacking the compound correlates with an increase in A3G CDA.

51. The method of any one of claims 34-50, wherein the method is high throughput.

52. The method of any one of claims 34-51, wherein the oligonucleotide having the CCC sequence or CC sequence comprises:
GATTGGTTGGTTATTTGTTTAAGGAAGGTGGATTAAAGAGAGTTAGAATG
TAGGAGTGGTATAGGAGTAATTGAATGATGATAGGTATGGAATAGTAGTT
GATTAAAGGCCCAATAAGGTGATGGAAGTTATGTTTGGTAGATTGATGG.

53. The method of any one of claims 34-52, wherein the oligonucleotide having the CCU sequence or CU sequence comprises:
GGATTGGTTGGTTATTTGTTTAAGGAAGGTGGATTAAAGAGAGTTAGAAT
GTAGGAGTGGTATAGGAGTAATTGAATGATGATAGGTATGGAATAGTAGT
TGATTAAAGGCCCAATAAGGTGATGGAAGTTATGTTTGGTAGATTGATG
G.

54. The method of any one of claims 34-53, wherein the mixture includes about 200 nM of A3G, about 150 nM of Vif, and about 1 fmole of the oligonucleotide having a CCC sequence or a CC sequence.

55. The method of any one of claims 34-53, wherein the mixture includes about 50 nM to about 300 nM of A3G.

56. The method of any one of claims 34-53, wherein the mixture includes about 100 nM to about 250 nM of A3G.

57. The method of any one of claims 34-53, wherein the mixture includes about 200 nM of A3G.

58. The method of any one of claims 34-53, wherein the mixture includes 50 nM to 250 NM of Vif.

59. The method of any one of claims 34-53, wherein the mixture includes 100 nM to about 200 nM of Vif.

60. The method of any one of claims 34-53, wherein the mixture includes 150 nM of Vif.

61. The method of any one of claims 34-53, wherein the mixture includes about 0.01 to about 0.5 fmole of oligonucleotide having a CCC sequence or CC sequence.

62. The method of any one of claims 34-53, wherein the mixture includes about 0.05 to about 0.3 fmole of oligonucleotide having a CCC sequence or CC sequence.

63. The method of any one of claims 34-53, wherein the mixture includes about 0.1 of oligonucleotide having a CCC sequence or CC sequence.

64. An assay for identifying a compound that has an inhibitory effect on Vif, comprising:
providing a mixture comprising
an amount of A3G,
an amount of Vif, and
an amount of an oligonucleotide having a CCC sequence or CC sequence;
contacting the mixture with a compound to form a sample;
measuring a conversion of the oligonucleotide having a CCC sequence or CC sequence in the sample to an oligonucleotide having a CCU sequence or CU sequence; and
determining the compound is capable of inhibiting Vif function based on the measurement of the conversion of the oligonucleotide having a CCC sequence or CC sequence to the oligonucleotide having the CCU sequence or CU sequence.

65. A kit for identifying a compound that inhibits Vif, comprising:
an amount of A3G,
an amount of Vif, and
an amount of an oligonucleotide having a CCC sequence or CC sequence.

66. The method of any one of claims 38-63, wherein SEQ ID NO: 4 comprises a mismatched nucleotide adjacent to the adenine at the 3′-end.

67. The method of any one of claims 38-63, wherein SEQ ID NO: 5 comprises a mismatched nucleotide two bases away from the adenine at the 3′-end.
